(19) United States
(12) Patent Application Publication
(10) Pub. No.: US 2016/0256571 A1
(43) Pub. Date: Sep. 8, 2016

(54) Invention

(71) Applicants: SANOFI, Paris (FR); UNIVERSITE PIERRE ET MARIE CURIE (PARIS 6), Paris (FR); CENTRE NATIONAL DE LA RECHERCHE SCIENTIFIQUE (C.N.R.S.), Paris (FR); INSTITUT NATIONAL DE LA SANTE ET DE LA RECHERCHE MEDICALE (INSERM), Paris (FR)

(72) Inventors: Marisol CORRAL-DEBRINSKI, MONTREUIL (FR); Christopher LECHAUVE, BAGNEUX (FR); Jose-Alain SAHEL, PARIS (FR); Thomas DEBEIR, PARIS (FR)

(21) Appl. No.: 14/389,368

(22) PCT Filed: Sep. 30, 2013
(86) PCT No.: PCT/IB2013/002461

§ 371 Date: Sep. 29, 2014

(51) Int. Cl. A61K 48/00; A61K 38/17

(52) U.S. Cl. CPC A61K 48/0066; A61K 38/1722

(57) ABSTRACT

The present invention concerns a neuroglobin agonist for use in the treatment or prevention of a mitochondrial disease associated with respiratory chain complex I (RCCI) deficiency and/or respiratory chain complex III (RCCIII) deficiency.

The present invention concerns a neuroglobin agonist for use in the treatment or prevention of a mitochondrial disease associated with respiratory chain complex I (RCCI) and/or respiratory chain complex III (RCCIII) deficiency.

### BACKGROUND TO THE INVENTION

Neuroglobin (NGB) was identified in vertebrates as a member of the globin superfamily. The protein is highly abundant in different brain regions and in the eye (Burmester T. et al. (2000) Nature 407: 520-523). NGB is now considered as a neuroprotectant under hypoxia or oxidative stress (Li R C. Et al. (2010) J Cereb Blood Flow Metab 30: 1874-1882; Hummler N, et al. (2013) Exp Neurol 236: 112-121). NGB expression is correlated to numerous pathologies such as Glaucoma or Alzheimer disease (Rajendram R, Rao N A (2008) Br J Ophthalmol 91: 663-666). The evidence linking NGB and mitochondrial function has increased in the last years (Liu J, et al. (2009) J Neurosci Res 87: 164-170; Yu Z, et al. (2012) Neuroscience 218: 235-242). However, the molecular mechanism by which NGB would regulate or affect mitochondrial function under normal or pathologic conditions remains to be elucidated. Indeed, in vivo models overexpressing or underexpressing NGB protein have not permit to clearly elucidate NGB function in mitochondria and notably its implication in pathogenesis of mitochondrial disorders (Khan A A, Gene. 2007 Aug. 15; 398(1-2):172-6; Hundahl C A, PLoS One. 2011; 6(12):e28160).

Mitochondrial disorders represent a common cause of chronic morbidity and are more prevalent than previously thought; indeed as a group, mitochondrial disorders affect at least 1 in 5,000 individuals (Schaefer A M, et al. (2008) Ann Neurol 63: 35-39). This high incidence of mitochondrial diseases in the population spotlights the essential role of mitochondria in energy production, reactive oxygen species (ROS) biology, apoptosis, and intermediate metabolic pathways. An array of mitochondrial diseases has been linked to respiratory chain complex I (RCCI) or complex III (RCCIII) deficiency. These mitochondrial diseases associated with RCCI or RCCIII deficiency represent a heterogeneous group of neuromuscular and multisystemic disorders of variable severity that are present in childhood and adulthood. Indeed, up to date, molecular defects observed in both mitochondrial DNA-encoded and nuclear DNA-encoded genes of mitochondrial proteins are associated with a wide spectrum of clinical problems including myopathy, encephalomyopathy, gastrointestinal syndromes, dystonia, diabetes, blindness, deafness and cardiomyopathy. Additionally, mitochondrial impairment is a key player in the pathogenesis process of Glaucoma, Alzheimer and Parkinson diseases (Coskun P, et al. (2012) Biochim Biophys Acta 1820: 553-564). Despite the huge advances in the understanding of molecular and biochemical bases underlying mitochondrial dysfunction, the ability to counteract mitochondrial pathologies and notably, mitochondrial diseases associated with RCCI or RCCIII deficiency, remains very limited (Pfeffer G et al., Cochrane Database Syst Rev. 2012 Apr. 18; 4:CD004426)

The inventors determined that NGB localizes to the mitochondria in rat and mouse retinas and that NGB expression knockdown provokes rat retinal ganglion cell (RGC) degeneration and RCCI and RCCIII defects in optic nerves that engender visual function impairment.

Using a mouse model exhibiting the main features of human neurodegenerative diseases due to RCCI deficiency, such as degeneration of the cerebellum, retina, optic nerve, thalamic, striatal, and cortical regions (Klein et al., Nature. 2002 Sep. 26; 419 (6905):367-74), the inventors have found out that NGB expression is decreased in the retina due to a reduction in both the number of NGB-positive cells and the overall NGB expression both at the mRNA and the protein levels. The inventors further demonstrated that overexpression of NGB protein in neuronal cells affected with RCCI deficiency, prior significant development of injuries, prevented these cells from undergoing degeneration, without noticeable side-effects. Importantly, preservation of retinal nerve fibers due to NGB overexpression in RGC resulted into maintenance of visual acuity of mice. These results demonstrate that NGB overexpression in a mouse model of human neurodegenerative disease due to RCCI deficiency translated into preservation of the visual function.

Altogether, in light of the relationship between NGB expression knockdown and RCCI and RCCIII defects identified by the inventors, these results demonstrate that an NGB agonist, by inducing NGB overexpression, can be used for the prevention or treatment of a mitochondrial diseases in patients having RCCI deficiency and/or RCCIII deficiency.

### BRIEF SUMMARY OF THE INVENTION

The invention concerns a neuroglobin (NGB) agonist for use in the treatment or prevention of a mitochondrial disease associated with RCCI deficiency and/or RCCIII deficiency.

The invention further concerns a method for preventing or treating a mitochondrial disease associated with RCCI deficiency and/or RCCIII deficiency in a subject having or at risk of having such disorder comprising administration of a therapeutically effective amount of a NGB agonist to the subject.

The invention also concerns a method for restoring or improving RCCI and/or RCCIII function in a subject having or at risk of having a mitochondrial disease associated with RCCI deficiency and/or RCCIII deficiency, comprising administration of a therapeutically effective amount of a neuroglobin agonist to the subject.

The inventors previously demonstrated that Harlequin (Hq) mice 6-9 months old exhibit up to 36% of RGC loss compared with control mice that correlated with the disappearance of optic fibres and with a severe defect of RCCI in optic nerves. The Hq mouse strain is an in vivo model of human neurodegenerative diseases due to RCCI deficiency caused by the knockdown of the nuclear gene encoding the mitochondrial Apoptosis Inducing Factor (AIF). The inventors evidenced that the intravitreal administration of the AAV2/2-AIF in Hq mice aged between 4-6 weeks was successful in preventing optic atrophy. Undeniably, RGC loss was prevented since eyes treated with the vector had a RGC population which attained ˜89% of control value. Moreover, in optic nerves from treated eyes, RCCI activity reached 81% of the control value.

The inventors have demonstrated that a reduction in NGB expression in rat primary cultured cells induces a significant defect in RCCI and RCCIII activities. The inventor has hypothesized that NGB activity could be linked to RCCI and RCCIII activities in vivo and have surprisingly found that in vivo, NGB activity is not only linked to RCCI and RCCIII activities but can also rescue their dysfunction. Indeed, by using an in vivo model of RCCI mitochondrial disease (the Hq mouse strain), bearing a wild type NGB gene and a mutated AIF gene, the inventors have demonstrated that the overexpression of NGB in vivo rescues RCCI dysfunction.

The inventors have now unexpectedly demonstrated that respiratory chain complexes activity, in particular RCCI activity, correlates with NGB expression in Hq mice. Moreover, the inventors have shown that NGB overexpression was effective in: (1) improving retinal ganglion cells (RGC) survival; (2) preserving nerve fiber integrity; (3) rescuing RCCI dysfunction; (4) protecting visual function and thus treating and preventing retinal damages induced by respiratory chain complex deficiency in particular RCCI deficiency, in this mitochondrial deficiency model.

The inventors have also demonstrated that NGB overexpression in Hq eyes leads to an increase of 38% in RCCI activity in optic nerves, thus reaching 78% of the control value; the increased RGC viability is the consequence of the protection of respiratory chain function since the population attains 75% of the value measured in control mice. Accordingly, NGB overexpression confers long-lasting visual function preservation; indeed, at the time of vector administration, Hq mice exhibited a visual behavior almost identical to control mice but by the age of 6 months the visual acuity of untreated Hq mice declines inexorably; events almost completely prevented by NGB overexpression.

Hence, inventor results show that overtime vision deteriorates in untreated eyes of Hq mice while eyes treated with the vector exhibited visual acuities comparable to control mice with an increase in their scores of 2-fold relative to untreated eyes. Indeed, the inventors demonstrated that NGB overexpression holds back RGC death and optic atrophy.

Altogether, these results show for the first time that NGB can be used as a therapeutic approach in treating or preventing RCCI and RCCIII deficiency in mitochondrial disease conditions.

### DETAILED DESCRIPTION OF THE INVENTION

The invention concerns a NGB agonist for use in the treatment or prevention of a mitochondrial disease associated with RCCI deficiency and/or RCCIII deficiency.

The invention further concerns a method for preventing or treating a mitochondrial disease associated with RCCI deficiency and/or RCCIII deficiency in a subject having or at risk of having such disorder comprising administration of a therapeutically effective amount of a NGB agonist to the subject notably, to increase the expression or activity of NGB protein in said subject and notably in target cells of said subject.

The invention further concerns a NGB agonist for use in the treatment or prevention of a RCCI and/or RCCIII deficiency in a patient.

As used herein, the term “Mitochondrial disease” refers to disorders in which deficits in mitochondrial respiratory chain activity contribute in the development of pathophysiology of such disorders in a mammal. Mitochondrial disorders may be caused by mutations, acquired or inherited, in mitochondrial DNA (mtDNA) or in nuclear genes that code for mitochondrial components. They may also be the result of acquired mitochondrial dysfunction due to adverse effects of drugs, infections, or other (environmental . . . ) causes.

As used herein, the term “a mitochondrial disease associated with respiratory chain complex I deficiency” or “a mitochondrial disease associated with RCCI deficiency” refers to a mitochondrial disease in which a dysregulation, a reduction or an abolition of RCCI complex activity is observed. The term “mitochondrial disease associated with RCCI deficiency” also refers to a mitochondrial disease induced by RCCI deficiency or in which RCCI deficiency increases the risk of developing such mitochondrial disease.

Examples of mitochondrial diseases associated with RCCI deficiency may be Leber's hereditary optic neuropathy (LHON), MELAS (Mitochondrial encephalomyopathy, lactic acidosis, and stroke-like episodes), MERRF (Myoclonic Epilepsy with Ragged Red Fibers), Leigh Syndrome (LS), and Fatal infantile multisystem disorder (for review see Papa and De Rasmo Trends in Molecular Medicine, 2013, Vol. 19, No. 1: 61-69 and http://www.mitomap.org/MITOMAP).

As used herein, the term “respiratory chain complex I” or “RCCI” refers to a protein complex located in the mitochondrial inner membrane that forms part of the mitochondrial respiratory chain. RCCI contains about 45 different polypeptide subunits, including NADH dehydrogenase (ubiquinone), flavin mononucleotide and several different iron-sulfur clusters containing non-heme iron. The iron undergoes oxidation-reduction between Fe(II) and Fe(III), and catalyzes proton translocation linked to the oxidation of NADH by ubiquinone. RCCI is also named NADH:quinone oxidoreductase (E.C. 1.6.5.3).

The RCCI function or RCCI activity may be measured by: (1) a very accurate and powerful spectrophotometric assay designed for minuscule biological samples (Benit et al., Clinica Chimica Acta 374 (2006) 81-86); (2) the biochemical analysis of respiratory chain (oxidative phosphorylation) complexes using Blue native (BN) polyacrylamide gel electrophoresis (PAGE) after the extraction from tissues or cells of enriched mitochondrial membranes; both the in-gel activity of respiratory chain complexes and the protein composition of each one of them could be analyzed (Calvaruso et al., Methods 46 (2008) 280-286).

As used herein, the term “a mitochondrial disease associated with respiratory chain complex III deficiency” or “a mitochondrial disease associated with RCCIII deficiency” refers to a mitochondrial disease in which a dysregulation, a reduction or an abolition of RCCIII complex activity is observed. The term “mitochondrial disease associated with RCCIII deficiency” also refers to a mitochondrial disease induced by RCCIII deficiency or in which RCCIII deficiency increases the risk of developing such mitochondrial disease.

Examples of mitochondrial diseases associated with RCCIII deficiency may be Encephalopathy, hepatic failure and tubulopathy, Leigh Syndrome, GRACILE syndrome, Bjornstad Syndrome, Hypoglycemia, Lactic acidosis and severe neurological phenotype (for review see Benit et al., Biochimica et Biophysica Acta 1793 (2009) 181-185; http://www.mitomap.org/MITOMAP).

As used herein, the term “respiratory chain complex III” or “RCCIII” refers to a protein complex located in the mitochondrial inner membrane that forms part of the mitochondrial respiratory chain. RCCIII contains about 11 polypeptide subunits including four redox centers: cytochrome b/b6, cytochrome c1 and a 2Fe-2S cluster. RCCIII function is to catalyze the oxidation of ubiquinol by oxidized cytochrome c1. RCCI is also named bc1 complex; ubiquinol cytochrome c reductase (EC 1.10.2.2).

The RCCIII function or RCCIII activity may be measured by: (1) a very accurate an powerful spectrophotometric assay designed for minuscule biological samples (Befit et al., Clinica Chimica Acta 374 (2006) 81-86); (2) the biochemical analysis of respiratory chain (oxidative phosphorylation) complexes using Blue native (BN) polyacrylamide gel electrophoresis (PAGE) after the extraction from tissues or cells of enriched mitochondrial membranes; both the in-gel activity of respiratory chain complexes and the protein composition of each one of them could be analyzed (Calvaruso et al., Methods 46 (2008) 280-286).

In one embodiment, the NGB agonist may be used for restoring or improving RCCI and/or RCCIII function in cells, in particular in neuronal cells.

In one embodiment, the mitochondrial disease associated with RCCI deficiency and/or RCCIII deficiency is a neurodegenerative disease or an ocular disease.

In one embodiment, the mitochondrial disease associated with RCCI deficiency and/or RCCIII deficiency is a mitochondrial disease associated with NGB expression and/or activity deficiency.

As used herein, the term “neurodegenerative disease” refers to all and any disease where a progressive loss of structure or function of neurons, including death of neurons, occurs. Such neurodegenerative disease may be for example Alzheimer disease, Parkinson's disease, Huntington disease or Amyotrophic lateral sclerosis.

As used herein, the term “ocular disease” refers to a disease, disorder, or abnormality that relates to the state of the eye, particularly the ocular disease may be a retinal disease or an optic neuropathy.

As used herein, the term “optic neuropathy” refers to damage to the optic nerve which induces degenerescence of the optic nerve; the optic nerve is composed of the retinal ganglion cell axons or nerve fibers (long slender projection of the nerve cell body which conducts electrical impulses originated form retinal neuron light stimulation which will be transmitted to the visual cortex), astrocytes (glial cells involved in biochemical support, repair and scarring processes) and oligodendrocytes (synthesis of the myelin sheath for insulating the axons).

As used herein, the term “retinal disease” refers to a disease, disorder, or abnormality that relates to retina. The retinal disease may have environmental or genetic origins. The term “retinal disease” also refers to a retinal degenerative disease implicating one or more of the retinal ganglion cells (RGCs), the photoreceptor cells, the horizontal cells, the bipolar cells, the amacrine cells and the optic nerve fibers. The retinal disease may be age-related macular degeneration, retinitis pigmentosa, diabetic retinopathy, glaucoma, or optic atrophy.

In one embodiment, the mitochondrial disease associated with RCCI deficiency and/or RCCIII deficiency is a mitochondrial disease associated with Apoptosis Inducing Factor (AIF) deficiency.

“AIF” or “Apoptosis Inducing Factor” is a protein that triggers chromatin condensation and DNA degradation in a cell in order to induce programmed cell death. AIF (Acc No AAV54054.1) also plays an important role in regulation of mitochondrial morphology and energy metabolism and has been proposed to regulate the respiratory chain indirectly, through assembly and/or stabilization of complexes I and III. It has been shown that AIF silencing induces decrease in complexes I and III activity. A crucial role of the AIF redox activity for normal mitochondrial functioning is evidenced by the fact that only expression of full-length AIF can restore defects in complex I and the cell growth supportive function in AIF deficient cells (AIF-/y cells) (for review Sevrioukova I F. Antioxid Redox Signal. 2011 Jun. 15; 14(12):2545-79). The Harlequin (Hq) mutation is a proviral insertion in the AIF gene, causing about a 90% reduction in AIF expression. The Harlequin mouse strain exhibits the main features of human neurodegenerative diseases due to RCCI deficiency, such as the degeneration of the cerebellum, retina, optic nerve, thalamic, striatal, and cortical regions.

“AIF deficiency” means the negative alteration of AIF expression or biological activity. The AIF expression or biological activity may be altered due for example, to a mutation or a deletion of the AIF gene or a mislocalization of the corresponding protein, a dysregulation of AIF protein or an underexpression of AIF protein.

A mitochondrial disease associated with an AIF deficiency according to the invention may be X-linked mitochondrial encephalopathy or an oxidative phosphorylation (OXPHOS) disease. The OXPHOS system consists of five mitochondrial inner membrane embedded multisubunit complexes: complex I (CI or NADH:ubiquinone oxidoreductase; EC 1.6.5.3), complex II (CII or succinate:ubiquinone oxidoreductase; EC 1.3.5.1), complex III (CIII or ubiquinol:cytochrome c oxidoreductase; EC 1.10.2.2), complex IV (CIV or cytochrome-c oxidase; EC 1.9.3.1) and complex V (CV or FoF1-ATP-synthase; EC 3.6.1.34). These complexes are divided into two functional parts: (i) the four complexes (CI-CIV) of the electron transfert chain and (ii) CV that generates ATP (for review see Koopman et al., The EMBO Journal; 2013-32: 9-29).

In one embodiment, the mitochondrial disease associated with RCCI and/or a RCCIII deficiency is a mitochondrial disease (optionally, associated with an AIF deficiency) wherein said mitochondrial disease is not associated with (or caused by) a mutation or deletion of the NGB gene or wherein said mitochondrial disease is not induced by NGB deficiency.

As used herein, the term “NGB deficiency” means the negative alteration of NGB expression or its biological activity. NGB expression or biological activity may be altered due for example to a mutation or a deletion of the NGB gene or a mislocalization of the NGB protein, a dysregulation of the NGB protein or an underexpression of the NGB protein.

As used herein, the term “target cells” refers to the cells of interest having a complex I or III deficiency. The term “target cells” also refers to the cells in which the expression and/or activity of NGB is to be increased. Target cells may be neurons, glial cells (such as astrocytes or oligodendrocytes) or retinal cells, notably, Retinal Ganglion Cells (RGCs).

As used herein, the term “treat,” “treatment,” or “treating” refers to any method used to partially or completely alleviate, relieve, inhibit, and/or reduce incidence of one or more symptoms or features and/or extending the lifespan of an individual suffering from a mitochondrial disease, disorder, and/or condition. In some embodiments, treatment may be administered to a subject who exhibits only early signs of the mitochondrial disease, disorder, and/or condition for the purpose of decreasing the risk of developing pathology associated with the mitochondrial disease, disorder, and/or condition.

As used herein, the term “prevent,” “prevention,” or “preventing” refers to any method to partially or completely prevent or delay the onset of one or more symptoms or features of a mitochondrial disease. Prevention may be administered to a subject who does not exhibit signs of a mitochondrial disease.

The “subject” or “individual” may be, for example, a human or non human mammal, such as a rodent (mouse, rat), a feline, a canine or a primate, affected by or likely to be affected by a mitochondrial disease. Typically, the subject is a human.

As used herein, the term “neuroglobin protein” or “NGB protein” encompass any naturally occurring isoform of the neuroglobin protein, including the protein of SEQ ID NO: 1, allelic variants thereof, splice variants thereof and orthologous proteins. NGB protein is highly conserved among vertebrates. Typically, NGB protein may be from various species such as for example, mammalian, avian, reptilian or amphibians. In the context of the invention, the man skilled in the art will readily determine the appropriate NGB orthologous protein (or the polynucleotide encoding for such NGB protein) to be used according to the patient to be treated. Typically, the NGB protein (or the polynucleotide encoding for such NGB protein) may be from the same species than the patient to which it is administered. Typically, NGB protein may be the human NGB (Accession Number NP_067080.1) having the sequence of SEQ ID NO: 1. Human NGB protein is encoded by the polynucleotide of sequence SEQ ID NO: 2.

As used herein, the term “neuroglobin agonist” or “NGB agonist” refers to a compound that induces or increases NGB biological activity. The biological activity of NGB depends on the amount of the protein (i.e. its expression level) as well as on the activity of the protein. Therefore, the NGB agonist may increase the expression or activity of NGB protein in target cells.

Methods for determining whether a compound is a NGB agonist are well-known by the person skilled in the art. For example, the person skilled in the art can assess whether a compound induces NGB expression.

According to the invention, the “level of expression of Neuroglobin” is determined by detecting a nucleic acid comprising SEQ ID NO: 2, a variant, a fragment, a complementary sequence or a corresponding RNA sequence thereof.

Level of expression of a gene or a nucleic acid can be performed by methods which are well known to the person skilled in the art, including in particular direct hybridization based assays and amplification-based assays.

The methods using direct hybridization based assays refer to pairing and binding of a nucleotide sequence (probe) to a complementary sequence to Neuroglobin messenger RNA (mRNA) or transcript and cDNA. The probe is designed using partial or full NGB nucleotide sequence. For example, such probe may be one or more of sequence sequences SEQ ID NO: 10 and/or, SEQ ID NO: 11, SEQ ID NO: 24, SEQ ID NO: 25. The quantification of NGB transcript expression utilizes methods well known in the art such as nucleic acid arrays, RNase protection assays, Northern-Blots, Slot-Blots or other technologies. The resulting complexes from hybridization are quantified by the nucleotide probes by well known technologies in the art such as fluorescence, luminescence, radio element labeling or other technologies.

The methods using amplification-based assays refer to technologies amplifying a specific transcript such as NGB transcript (precursor/mRNA/cDNA) using methods, well known in the art, such as Polymerase Chain Reaction (PCR). The PCR technology uses, among others components, specific primers (direct and forward) designed using the nucleotide sequences of the NGB transcript to amplify partially or fully NGB nucleotide sequence. For example, such primers may be of sequence one or more of sequences SEQ ID NO: 10 and/or, SEQ ID NO: 11, SEQ ID NO: 24, SEQ ID NO: 25. Quantification uses nucleotide probes by well known technologies in the art such as fluorescence, luminescence, radio element labeling and other technologies. A representative technology is the TaqMan technique developed by, among other companies, Applied Biosystems (Perkin Elmer) in which a specific transcript as NGB is quantified by the release of a fluorescent reporter dye. The fluorescent reporter dye is release from a specific hybridization probe in real-time during a polymerase chain reaction (PCR) and is proportional to the accumulation of the PCR product.

In certain embodiments, a “gene” refers to a nucleic acid that is transcribed. In certain aspects, the gene comprises a nucleic acid, and/or encodes a polypeptide. In this respect, the term “gene” is used for simplicity to refer to a nucleic acid comprising a nucleotide sequence that is transcribed, the corresponding sequence in RNA bases and the complement thereof. In particular aspects, the transcribed nucleotide sequence comprises at least one functional protein, polypeptide and/or peptide encoding unit. As will be understood by those in the art, this functional term “gene” includes both genomic sequences, RNA (mRNA, Long intergenic non-coding RNAs . . . ) or cDNA sequences, or smaller engineered nucleic acid segments.

The term “nucleic acid” or “polynucleotide” will generally refer to at least one molecule or strand of DNA, RNA or a derivative or mimic thereof, comprising at least one nucleobase, such as, for example, a naturally occurring purine or pyrimidine base found in DNA (e.g., adenine “A,” guanine “G,” thymine “T,” and cytosine “C”) or RNA (e.g. A, G, uracil “U,” and C). The term “nucleic acid” encompasses the terms “oligonucleotide” and “polynucleotide.” The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule that comprises one or more complementary strand(s) or “complement(s)” of a particular sequence comprising a strand of the molecule.

A nucleic acid may be made by any technique known to one of ordinary skill in the art. Non-limiting examples of synthetic nucleic acid, particularly a synthetic oligonucleotide, include a nucleic acid made by in vitro chemical synthesis using phosphotriester, phosphite or phosphoramidite chemistry and solid phase techniques via deoxynucleoside H-phosphonate intermediates such described by Froehler et al., 1986 Nucleic Acids Res. 1986 Jul. 11; 14(13):5399-407. A non-limiting example of enzymatically produced nucleic acid include one produced by enzymes in amplification reactions such as PCR™ or the synthesis of oligonucleotides. A non-limiting example of a biologically produced nucleic acid includes recombinant nucleic acid production in living cells (see for example, Molecular cloning: a laboratory manual.—4th ed./Michael R. Green, Joseph Sambrook. 2012 Cold Spring Harbor, N.Y.). A nucleic acid may be purified on polyacrylamide gels, cesium chloride centrifugation gradients, or by any other means known to one of ordinary skill in the art (see for example, Molecular cloning: a laboratory manual.—4th ed./Michael R. Green, Joseph Sambrook. 2012 Cold Spring Harbor, N.Y.). The nucleic acid molecule may be isolated, which means that it is essentially free of other nucleic acids. Essentially free from other nucleic acids means that the nucleic acid molecule is at least about 90%, typically at least about 95% and, and notably at least about 98% free of other nucleic acids. In one embodiment, the molecule is essentially pure, which means that the molecule is free not only of other nucleic acids, but also of other materials used in the synthesis and isolation of the molecule. Materials used in synthesis include, for example, enzymes. Materials used in isolation include, for example, gels, such as SDS-PAGE. The molecule is at least about 90% free, typically at least about 95% free and, and notably at least about 98% free of other nucleic acids and such other materials.

The term “variants” includes protein and nucleic acid variants. Variant proteins may be naturally occurring variants, such as splice variants, alleles and isoforms. Variations in amino acid sequence may be introduced by substitution, deletion or insertion of one or more codons into the nucleic acid sequence encoding the protein that results in a change in the amino acid sequence of the protein. Variant proteins may be a protein having a conservative or non-conservative substitution. Variant proteins may include proteins that have at least about 80% amino acid sequence identity with a polypeptide sequence disclosed herein. A variant protein may have at least about 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length polypeptide sequence or a fragment of a polypeptide sequence as disclosed herein. Amino acid sequence identity is defined as the percentage of amino acid residues in the variant sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both. The percentage of identity for protein sequences may be calculated by performing a pairwise global alignment based on the Needleman-Wunsch alignment algorithm to find the optimum alignment (including gaps) of two sequences along their entire length, for instance using Needle, and using the BLOSUM62 matrix with a gap opening penalty of 10 and a gap extension penalty of 0.5.

Variant nucleic acid sequences may include nucleic acid sequences that have at least about 80% nucleic acid sequence identity with a nucleic acid sequence disclosed herein. A variant nucleic acid sequence may have at least about 80%, 81%, 82%, 83%, 84%, 85%, 86%, 87%, 88%, 89%, 90%, 91%, 92%, 93%, 94%, 95%, 96%, 97%, 98%, 99% nucleic acid sequence identity to a full-length nucleic acid sequence or a fragment of a nucleic acid sequence as disclosed herein. Nucleic acid sequence identity can be calculated by methods well-known to one of skill in the art. The percentage of identity may be calculated by performing a pairwise global alignment based on the Needleman-Wunsch alignment algorithm to find the optimum alignment (including gaps) of two sequences along their entire length, for instance using Needle, and using the DNAFULL matrix with a gap opening penalty of 10 and a gap extension penalty of 0.5.

The term “fragments” includes protein and nucleic acid fragments. A protein sequence may be truncated at the N-terminus or C-terminus, or may lack internal residues, for example, when compared with a full length protein. Preferably, said fragments are at least about 10, 20, 30, 40, 50, 60, 70, 80, 90, 100, 110, 120, 139, 149, 150 amino acids in length. For example, a NGB fragment may contain amino acids 1 to 149, 1 to 144, 5 to 149 or 5 to 144 of sequence SEQ ID NO: 1.

Level of expression of a protein or a polypeptide may be assessed by using immunologic methods such as detection using polyclonal or monoclonal antibodies, chimeric antibody or humanized antibodies. The level of expression of the NGB protein is quantified using technologies well known by the art.

Suitable immunologic methods include enzyme linked immunoassays (ELISA), sandwich, direct, indirect, or competitive ELISA assays, enzyme linked immunospotassays (ELIspot), radio immunoassays (RIA), flow-cytometry assays (FACS), immunohistochemistry, Western Blot, fluorescence resonance energy transfer (FRET) assays, protein chip assays using for example antibodies, antibody fragments, receptor ligands or other agents binding the NGB proteins.

Other methods for determining whether a compound is a NGB agonist may be for example, by measuring the biological activity of NGB, through measuring one of the phenomenon in which NGB is known to play a role. For instance, the inventors have demonstrated that NGB is implicated in mitochondrial complex I and III activity. Indeed, the biological activity of NGB protein and notably the increased of NGB protein biological activity, may be assessed through measuring the RCCI and RCCIII activity.

The biological activity of NGB may also be measured by assessing the capacity of NGB to bind to its natural binding partners such as cytochrome c (Cyt C), a small heme protein associated with the inner membrane of the mitochondrion which transfers electrons between Complexes III (Coenzyme Q-Cyt C reductase) and IV (Cyt C oxidase). The binding of NGB to Cyc may for example be assessed using a co-immunoprecipitation assay, a pull-down assay or the yeast two hybrid system (Y2H). A compound that improves binding of NGB to Cyt C or other of its partners could be defined as a NGB agonist. Moreover, affinity purification-mass spectrometry (APMS) based on the biochemical purification of proteins from cell extracts could be performed; since this strategy allows the identification of protein interactions under the physiological conditions (M. E. Sardiu and M. P. Washburn J Biol Chem. 2011 8; 286(27): 23645-51).

The NGB agonist may be for example, a drug, a nucleic acid or a polypeptide.

In one embodiment, the NGB agonist may be a drug (e.g. a chemical molecule or a small molecule) such as Deferoxamin (DFO, CAS Number 7278-84-4), hemin (CAS Number 86-11-3), cinnamic acid (CAS Number 63938-16-9) or valproic acid (CAS Number 99-66-1). For example, the NGB agonist may be the HIF prolyl hydroxylase inhibitor (CAS Number 385786-48-1) or the 17 β-Oestradiol ((17β)-estra-1,3,5(10)-triene-3,17-diol, CAS Number 50-28-2) wich induce NGB expression.

In another embodiment the NGB agonist is a nucleic acid. For example, the NGB agonist may be a nucleic acid which comprises a polynucleotide encoding NGB protein. Typically, the NGB agonist may be an expression cassette comprising said polynucleotide.

In another embodiment, the NGB agonist may be a polypeptide such as a dominant activated mutant of NGB, a wild-type NGB protein, a fragment or a peptidomimetic thereof.

In another embodiment, the NGB agonist may be a polypeptide such as a dominant activated mutant of hypoxia-inducible factor-1 alpha (HIF-1 alpha), a wild-type HIF-1 alpha protein (Acc No: AAC50152.1), a fragment or a peptidomimetic thereof.

As used herein the term “protein” or “polypeptide” refers to any chain of amino acids linked by peptide bonds, regardless of length or post-translational modification. Polypeptides include natural proteins, synthetic or recombinant polypeptides and peptides (i.e. polypeptides of less than 50 amino acids) as well as hybrid, post-translationally modified polypeptides, and peptidomimetic.

As used herein the term “peptidomimetic” refers to peptide-like structures which have non-amino acid structures substituted but which mimic the chemical structure of the peptide and retain the functional properties of the peptide such as for example, the NGB protein. Peptidomimetics may be designed in order to increase peptide stability, bioavailability, solubility, etc.

Typically, the NGB agonist is a polypeptide encoded by a nucleic acid. For example, said nucleic acid may be an expression cassette.

“Expression cassette” according to the invention refers to a linear or circular nucleic acid molecule. This expression cassette also refers to DNA and RNA sequences which are capable of allowing the production of a functional nucleotide sequence in a suitable host cell. Typically, the expression cassette comprises a polynucleotide encoding a mutant of NGB such as a dominant activated mutant of NGB, a wild-type NGB protein, or a fragment thereof, said polynucleotide being operatively linked to at least one transcriptional regulatory sequence. The polynucleotide may comprise the sequence SEQ ID NO: 2. Typically, the expression cassette comprises a polynucleotide encoding NGB protein, said polynucleotide being operatively linked to at least one transcriptional regulatory sequence for the expression of NGB protein in target cells, said at least one transcriptional regulatory sequence being 3′UTR and/or 5′UTR NGB sequences.

In another embodiment, the expression cassette may comprises a polynucleotide encoding a mutant of HIF-1 alpha such as a dominant activated mutant of HIF-1 alpha, a wild-type HIF-1 alpha protein, or a fragment thereof, said polynucleotide being operatively linked to at least one transcriptional regulatory sequence.

The expression cassette can also include sequences required for proper translation of the nucleotide sequence of interest. The expression cassette may additionally contain selection marker genes. Typically, the cassette comprises in the 5′-3′ direction of transcription, a transcriptional and translation initiation region, a polynucleotide encoding the NGB protein, a transcription and translation termination region functional in mammalian cells.

The expression cassette may also include a multiple cloning site.

In addition to the components mentioned above, the expression cassette of the present invention may comprise the components required for homologous recombination.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the term “transcriptional regulatory sequence”, “transcription regulatory sequence>> or “regulatory sequences” refers to nucleotide sequences influencing the transcription, RNA processing or stability, or translation of the associated (or functionally linked) nucleotide sequence to be transcribed. The transcriptional regulatory sequence may have various localizations with the respect to the nucleotide sequences to be transcribed. The transcriptional regulatory sequence may be located upstream (5′ non-coding sequences), within, or downstream (3′ non-coding sequences) of the sequence to be transcribed (e.g., polynucleotide encoding NGB protein). The transcription regulating nucleotide sequences may be selected from the group consisting of enhancers, promoters, translation leader sequences, introns, 5′-untranslated sequences (5′UTR), 3′-untranslated sequences (3′UTR), and polyadenylation signal sequences. They include natural and synthetic sequences as well as sequences, which may be a combination of synthetic and natural sequences. As is noted above, the term “transcriptional regulatory sequence” is not limited to promoters. However, transcriptional regulatory sequence of the invention may comprise at least one promoter sequence (e.g., a sequence localized upstream of the transcription start of a gene capable to induce transcription of the downstream sequences), and/or at least one 3′UTR and/or one 5′UTR. In one preferred embodiment the transcription regulating nucleotide sequence of the invention comprises the promoter sequence of the NGB gene and/or the native 3′UTR of NGB gene and/or native 5′UTR of NGB gene. Furthermore, a fragment of the NGB 3′UTR and/or of the NGB 5′UTR may also be employed.

As used herein, the term “Promoter” or “promoter sequence” refers to a DNA sequence in a gene, usually upstream (5′) to its coding sequence, which controls the transcription of the coding sequence such as the polynucleotide encoding NGB protein by providing the recognition for RNA polymerase and other factors required for proper transcription. For example, the promoter may be the NGB promoter, a variant or a fragment thereof, preferably, the human NGB promoter. Promoters may contain DNA sequences that are involved in the binding of protein factors which control the effectiveness of transcription initiation in response to physiological or developmental conditions. Typically, the NGB promoter may contain two GC boxes which are bound by Sp1 and Sp3 factors. According to the invention, the promoter sequence may also contain enhancer elements. An “enhancer” is a DNA sequence which can stimulate promoter activity. It may be an innate element of the promoter or a heterologous element inserted to enhance the level and/or tissue-specificity of a promoter. Typically, the promoter sequence of the invention is a ubiquitous promoter, a tissue-specific promoter or an inducible promoter. “Ubiquitous Promoters” refers to those that direct gene expression in all tissues and at all times. The ubiquitous promoter may be eukaryotic or viral promoters. In one embodiment, the promoter sequence is eukaryotic promoter selected from the group consisting of the chicken β-actin promoter (CBA), the composite CAG promoter (consisting of the CMV immediate early enhancer and the chicken β-actin promoter) and the human phosphoglycerate kinase 1 (PGK) promoter. According to another embodiment, the promoter sequence is a viral promoter such as the human cytomegalovirus (CMV) promoter. The term “Tissue-specific” promoters as referred to herein are those that direct gene expression almost exclusively in specific tissues, such as retina specific promoter or central nervous system specific promoter. A retina specific promoter may be selected form the group consisting of the RPE65 promoter, VDM2 promoter, OA1 promoter, human rhodopsin kinase (RK) promoter, bovine rhodopsin promoter (RHO) and mice opsin promoter (mOP). Typically, the promoter sequence is an “Inducible promoters” refers to those that direct gene expression in response to an external stimulus, such as light, heat-shock and chemical.

The “untranslated region” or “UTR” refers to either of the two regions immediately adjacent to the coding sequence on a strand of mature mRNA. When it is found on the 5′ side, it is called the 5′ UTR (or 5′ untranslated region), or if it is found on the 3′ side, it is called the 3′ UTR (or trailer sequence). As used herein, the term “3′UTR neuroglobin sequence” refers to the sequence of the 3′UTR of the NGB gene, such as for example, the human neuroglobin 3′UTR (SEQ ID NO: 3). As compared with the transcript of the human NGB gene (SEQ ID NO: 2), the human 3′UTR of the NGB gene is the 3′ extremity of sequence SEQ ID NO: 2 starting at position 831 (positions 831-1054 of SEQ ID NO: 2).

As used herein, the term “5′UTR neuroglobin sequence” refers to the sequence of the 5′UTR of the NGB gene, such as for example, the human neuroglobin 5′UTR (SEQ ID NO: 4). As compared with the transcript of the human NGB gene (SEQ ID NO: 2), the human 5′UTR of the NGB gene is the 5′ extremity of sequence SEQ ID NO: 2 starting at position 1 (positions 1-315 of SEQ ID NO: 2). It has been recently described that the transcription start site of the human NGB mRNA locates at −306 bp relative to the translation start codon ATG (W. Zhang et al., Biochimica et Biophysica Acta 1809 (2011) 236-244). Indeed, the human neuroglobin 5′UTR may be a sequence corresponding to position 69 to 375 of SEQ ID NO: 4 (position 69 to 375 of SEQ ID NO: 2).

In one embodiment, the expression cassette is comprised in an expression vector.

The term “vector” refers to a nucleic acid sequence capable of transporting into a cell another nucleic acid to which the vector sequence has been linked. The term “expression vector” includes any vector containing a gene construct or an expression cassette in a form suitable for expression by a cell. The “expression vector” may be any recombinant vector capable of expression of a NGB protein or fragment thereof. More particularly, the expression vectors used can be derived from bacterial plasmids, transposons, yeast episome, from insertion elements, from yeast chromosomal elements, from viruses such as an adeno-associated virus (AAV) vector, a lentivirus vector, a retrovirus vector, a replication competent adenovirus vector, a replication deficient adenovirus vector and a gutless adenovirus vector, a herpes virus vector, baculoviruses, blinked as SV40 virus, the vaccinia virus, fox pox viruses, pseudorabies viruses. AAV and lentivirus vectors have emerged as the vectors of choice for gene transfer to the central nervous system as they mediate efficient long-term gene expression with no apparent toxicity. Moreover, several clinical trials have shown that direct infusion of AAV2 vectors into brain parenchyma in humans is well tolerated (Bowers et al., Human Molecular Genetics, 2011, Vol. 20, Review Issue 1 R28-R41). Recombinant AAV are the most common vectors used in both basic science and translational studies in retinal diseases. Up to date four clinical trials involving the administration of AAV are ongoing and concern more than 200 participants. AAV, a helper-dependent single-stranded DNA parvovirus, has never been shown to cause disease in humans or animals. The vector is able to durably and efficiently induce gene expression in dividing or terminally differentiated cells. It has been proven to be well tolerated with benign immune response. Also, manipulation of the AAV capsid as well as promoters effectively modulates cellular tropism which is critical to the cell specificity of many eye diseases (K. Willett and J. Bennett, Front Immunol. 2013, 30; 4: 261). The expression cassette can be inserted into the expression vector by methods well known in the art.

The expression vector may include reporter genes. Examples of reporter genes encode luciferase, (green/red) fluorescent protein and variants thereof, like eGFP (enhanced green fluorescent protein), hrGFP (humanized recombinant green fluorescent protein), RFP (red fluorescent protein, like DsRed or DsRed2), CFP (cyan fluorescent protein), BFP (blue fluorescent protein), YFP (yellow fluorescent protein), β-galactosidase or chloramphenicol acetyltransferase, and the like. These sequences are selected depending on the host cell implemented.

According to one embodiment of the invention, the expression vector is a viral vector. The viral vector of the invention may be derived from retroviruses, herpes simplex viruses, adenoviruses or AAVs. According to the present invention, these vectors are particularly advantageous.

In one embodiment, the expression vector of the invention is an AAV vector comprising respectively the 5′ inverted terminal repeat (ITR5′) and 3′ inverted terminal repeat (ITR3′) sequences of the AAV, at the 5′ and 3′ ends of said expression cassette.

As used herein, the terms “AAV vector” or “AAV particle” or “AAV plasmid” refer to the nucleic acid derived from any adeno-associated virus vector or any vector derived from an adeno-associated virus.

The term “Terminal inverted repeat sequence” or “ITR” means the terminal inverted repeat sequences of palindromic 145 base-pairs (bp) flanked at the 5′ and 3′ AAV vector according to the invention. The ITRs sequences are essential for the integration, replication and packaging of the viral vector. AAV ITR's can be modified using standard molecular biology techniques. Accordingly, AAV ITRs used in the vectors of the invention need not have a wild-type nucleotide sequence, and may be altered, e.g., by the insertion, deletion or substitution of nucleotides. Indeed, the ITR5 ‘ and ITR3’ are not necessarily identical but are functional. “Functional ITR sequences” means ITR sequences that allow vector replication and packaging. Additionally, AAV ITRs may be derived from any of several AAV serotypes, including but not limited to AAV-1, AAV-2, AAV-3, AAV-4 or AAV-5.

In another embodiment, the expression cassette is in a viral particle. Typically, the expression cassette inserted into an expression vector which is packaged or encapsidated in a viral particle.

As used herein, the “viral particle” refers to the packaged or encapsidated viral vector that is capable of binding to the surface and entering inside the host cells. The techniques for isolating viral particles of this invention from host cellular constituents and eventually from other types of viruses (such as helper viruses) which may be present in the host cell, are known to those of skill in the art, and include, for example, centrifugation and affinity chromatography. Typically, the viral particle may be an AAV particle.

The expression “adeno-associated virus” or “AAV” or “AAV particle” means non-enveloped single-stranded DNA belonging to the family Parvoviridae virus and Dependovirus genus. Wild-type AAVs are low integrative viruses but not lytic and non-pathogenic to humans. They infect a wide variety of mitotic and quiescent cells but are dependent on a helper virus for their replication, such as adenovirus or herpes virus.

As used herein, the term “rAAV” refers to a recombinant AAV-nucleic acid molecule containing some AAV sequences, usually at a minimum the ITRs and some foreign or exogenous (i.e., non-AAV) DNA, such as the NGB nucleic acid sequence of the invention

As used herein, the term “serotype” refers to an AAV which is identified by and distinguished from other AAVs based on capsid protein reactivity with defined antisera. There are at least twelve known serotypes of human AAV, including AAV1 through AAV12; however additional serotypes continue to be discovered, and use of newly discovered serotypes are contemplated. For example, AAV2 serotype 2 (AAV2/2) is used to refer to an AAV which contains capsid proteins encoded from the cap gene of AAV2 and a genome containing 5′ and 3′ inverted terminal repeat (ITR) sequences from the same AAV2 serotype.

The virus particle serotype determines its tropism. AAV2 viral particle is particularly advantageous. For AAV2 infection, heparan sulfate proteoglycan and the extracellular domain of the laminin receptor (37/67 kDa) are supposed as the primary receptors. Moreover, αv65 integrin, Fibroblast Growth Factor Receptor 1 and the Hepatocyte Growth Factor Receptor c-Met are reported to act as coreceptors.

Typically, the capsid protein of the viral particle may comprise at least one tyrosine residue which is mutated to phenylalanine. For example, the capsid protein may be mutated by substitution of at least three tyrosine residues by phenylalanine residues. Mutation of the capsid proteins modifies viral tropism or increases the transduction efficiency of the rAAV vector and reduces host cell damage. Advantageously, the tyrosine 444 of the capsid is substituted by a phenylalanine residue. Typically, the vector is an AAV-2 Y444F.

In another embodiment, the expression vector may be a lentiviral vector comprising sufficient lentiviral genetic information to allow packaging of an RNA genome, in the presence of packaging components, into a viral particle capable of infecting a host cell (such as the target cells).

As used herein, the term “lentiviral vector” refers to a vector derived from (i.e., sharing nucleotides sequences unique to) a lentivirus. The term “lentiviral vector” also refers to a modified lentivirus having a modified proviral RNA genome which comprises a NGB polynucleotide sequence. According to the invention, the lentiviral vectors derivative from the human immunodeficiency virus (HIV).

In another embodiment, the expression cassette may be contained in a host cell. Typically, the expression cassette is inserted into an expression vector which is contained in a host cell.

The introduction of recombinant vector into a host cell can be performed according to methods well known in the art such as transfection techniques (calcium phosphate, electroporation), lipofection (liposomes, charged lipids) or viral infection (lentivirus, adenovirus, herpes virus, etc. . . . ) or by the use of nanoparticles. Generally, the vector and the cells are contacted in a suitable device (plate, dish, tube, pouch, etc. . . . ) for a period of time sufficient to allow introduction of the expression vector into the host cells. Typically, the vector is introduced into the cells by calcium phosphate precipitation, electroporation, or by using one or more transfection-facilitating compounds, such as lipids, polymers, liposomes and peptides, etc. . . . . Precipitation of calcium phosphate is particularly suitable. The cells are cultured in any suitable medium, such as RPMI (Roswell Park Memorial Institute medium), DMEM (Dulbecco/Vogt modified Eagle's minimal essential medium) or specific to a culture medium in the absence of fetal calf serum, etc. . . .

As used herein, a “host cell” refers to any cell that harbors, or is capable of harboring, the expression cassette of the invention, the expression vector of the invention, or the NGB nucleic acid sequence or any cell that express, or is capable of expressing NGB protein. The term “host cell” also refers to a cell that has been transformed, or is capable of transformation, by an exogenous nucleic acid molecule such as the isolated polynucleotide of the invention. Host cells containing the transformed polynucleotide are referred to as “transgenic” host cells. Said host cells may be used to obtain organisms which are not human or may be obtained from said organisms. The host cell may be a prokaryotic cell (bacteria or cyanobacteria) or a eukaryotic cell (e.g. fungi, algae, yeast, plant, mammalian or insect cells). Typically, a mammal cell may be a rodent (mouse, rat), a feline, a canine or a primate cell. A mammal cell may be selected from the group of cell lines, tissue, somatic cells, neuron or neuronal derived cells, retinal cells and glial cells.

The method or use of the invention may comprise the step of delivering NGB agonist to target cells of the subject, thereby preventing, ameliorating, or treating said mitochondrial disease. Optionally, when the NGB agonist is a nucleic acid which comprises an expression cassette comprising a polynucleotide encoding neuroglobin protein, said polynucleotide being operatively linked to at least one transcriptional regulatory sequence, said method or use further comprises the step of expressing said polynucleotide in said subject preferably, in the target cells of said subject.

For the purpose of the invention, the NGB agonist may be delivered to the target cells by any means. The term “delivering the NGB agonist to the target cell” refers to the administration of the NGB agonist to the patient under any appropriate form such as a pharmaceutical composition, and by any suitable route which facilitates the delivery of the NGB agonist into the target cells in which the NGB agonist will provide the desired therapeutic or preventive effect. The NGB agonist may be delivered to the target cell by a direct introduction into patients by injection notably, intravitreal injection, spray or other means.

As used herein, the term “pharmaceutical composition” refers to a preparation of one or more of the expression cassette, a vector comprising said expression cassette and a viral particle comprising said vector, with other chemical components such as physiologically suitable carriers and excipients. The purpose of a pharmaceutical composition is to facilitate administration of said expression cassette, vector and/or viral particle to an organism.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Typically, a pharmaceutical composition may comprise the NGB agonist of the invention and a pharmaceutical acceptable vehicle. For example, said pharmaceutical composition may comprise (i) a polypeptide such as a dominant activated mutant of NGB, a wild-type NGB protein, a fragment or a peptidomimetic thereof, or (ii) a polynucleotide such as a polynucleotide encoding said polypeptide, optionally inserted into an expression cassette or an expression vector, or contained in a viral particle and (iii) a pharmaceutical acceptable vehicle.

As used herein, the term “Pharmaceutical acceptable vehicle” refers to a diluent, adjuvant, excipient or carrier with which the expression cassette, the vector and/or the viral particle of the invention is administered.

Pharmaceutical compositions for use in accordance with the present invention thus may be formulated in conventional manner using one or more physiologically acceptable carriers (“Pharmaceutical acceptable vehicle”) comprising excipients and auxiliaries, which facilitate processing of the active compounds into preparations which, CaO be used pharmaceutically. Proper formulation is dependent upon the route of administration chosen. For injection and more specifically for intravitreal injection, the neuroglobin agonist of the invention may be formulated in aqueous solutions, for example in physiologically compatible buffers such as Hank's solution, Ringer's solution, or physiological saline buffer. A physiologically compatible buffers may be for example, Balanced Sterile Solution (BSS BV1) commercialized by Industria Farmaceutica Galenica Senese, S.R.L.

As used herein, the term “therapeutically effective amount” means an amount of a compound or composition comprising said, that activates or increases the expression of NGB without any toxic effects on the target cell. In certain embodiments, said compound or salt thereof increase the NGB expression by more than about 30%, 40%, 50%, 60%, 70%, 80%, 90%, 95%, 97%, 98%, 99%, or 99.9%.

Although having distinct significances, the terms comprising, “containing”, and “consisting of” were used in an interchangeable way in the description of the invention, and can be replaced one by the other.

The invention will be further described in view of the following examples.

### BRIEF DESCRIPTION OF THE FIGURES

FIG. 1: Schematic representation of mitochondrial extraction from rat retinas. Successive steps during the process of purification will lead to a mitochondrial enrichment in the ultimate fraction. Different samples were evaluated by western blot analysis for the presence of Ngb or mitochondrial proteins.

FIG. 2: In vivo impact of neuroglobin down regulation on NGB and CYGB mRNA RT-qPCR assays were performed with total RNAs from retinas (isolated from rats 3 months after the eye electroporation, ELP) to determine the steady-state levels of NGB and CYGB mRNAs. 18 control eyes, 8 eyes treated with scrambled shRNA and 10 eyes treated with anti-Ngb shRNA were evaluated (mean±S.E.M presented). Relative fold variations were calculated using the comparative ΔΔCt method and the mitochondrial ATP6 gene as a normalizing gene. Primers used for each gene are shown in Table 2.

FIG. 3: In vivo impact of neuroglobin down regulation on SNCG and BRN3A mRNAs RT-qPCR assays were performed with total RNAs from retinas (isolated from rats 3 months after the ELP) to determine the steady-state levels of SNCG and BRN3A mRNAs. 18 control eyes, 8 eyes treated with scrambled shRNA and 10 eyes treated with anti-Ngb shRNA were evaluated (mean±S.E.M presented). Relative fold variations were calculated using the comparative ΔΔCt method and the mitochondrial ATP6 gene as a normalizing gene. Primers used for each gene are shown in Table 2.

FIG. 4: Detection of respiratory chain complex activities in optic nerves Illustrative curve for the successive measurements of complex I (EC 1.6.5.3) and complex V activities (EC 3.6.3.14); complex IV (CIV) (EC 1.9.3.1), complex II+III (CII+CIII) (EC 1.3.2.2) and complex III (CIII) (EC 1.10.2.2).

FIG. 5: Assessment of visual performance after neuroglobin knockdown Four different groups of rats were evaluated for head tracking movements at an angular speed identical to that of the drum rotation: 22 animals 8 week-old (before the ELP) and 10 control rats 18 week-old. No significant difference was measured in the three grating frequencies monitored in any of the animals examined in both clockwise and counterclockwise directions of motion.

FIG. 6: Assessment of visual performance after neuroglobin knockdown 22 rats treated with anti-Ngb shRNA, 20 rats treated with scrambled shRNA. An unequivocal and significant decline in visual performance was measured in rats treated with anti-Ngb shRNA which responded with poor clockwise scores. Rats treated with scrambled shRNA showed no significant difference between clockwise and counterclockwise responses. Mean values±S.E.M are presented.

FIG. 7: Cellular distribution and relative amounts of NGB in retinas from Harlequin and control retinas: RT-qPCR assays were performed using total RNAs from retinas (isolated from mice 6 months old) to determine the steady-state levels of NGB mRNA. RNAs purified from 31 control and 37 Hq retinas were evaluated. Relative fold mRNA variations were calculated using the comparative ΔΔCt method and the mitochondrial ATP6 gene as a normalizing gene and are represented relative to the value assessed in RNAs from control retinas considered as 1; each value is the mean of all the assessments±S.E.M. Control and Harlequin NGB mRNA abundance were compared with the unpaired non parametric significance test of Mann-Whitney. Primers used for the NGB gene are shown in Table 7.

FIG. 8: Physical Map of the AAV2/2-NGB vector genome (7255 bp), encompassing mouse NGB sequences inserted into the pAAV-IRES-hrGFP plasmid: The NGB ORF (453 bp) encoding 151 amino acids is in frame with three FLAG epitopes and transcribed under the control of the cytomegalovirus promoter (pCMV). The construction contains both UTRs (UnTranslated Regions) at the 5′ (279 bp) and 3′ (895 bp) ends of the mouse NGB mRNA (NM_022414.2). The plasmid possesses also a cassette allowing the expression of the recombinant humanized green fluorescent protein (hrGFP).

FIG. 9: AAV2/2-NGB vector generation and administration in Harlequin eyes: RT-qPCR assays were performed with total RNAs extracted from control retinas and 14 pairs of retinas isolated from Hq mice in which one eye was subjected to intravitreal injection of AAV2/2-NGB (mice were euthanized between 5 to 6 months after vector administration). The steady-state levels of NGB and AIF1 mRNAs were compared to those obtained in RNA preparations from 18 age-matched control mice. Two pairs of specific primers were used for NGB mRNA: one recognized the two NGB transcripts (the endogenous and the one issued from the vector) and the second pair recognized exclusively the molecule transcribed from the vector since it is located between the end of the ORF and the Flag epitope sequence (Table 7, NGB-AAV). Histograms show the steady-state levels of AIF1 mRNA and of NGB mRNAs calculated by the comparative ΔΔCt method and the mitochondrial ATP6 gene as a normalizing gene. Values shown are relative to the mRNA amounts measured in control retinas and considered as 1; each value is the mean of all the triplicates obtained from each biological sample±S.E.M. Statistical significance was determined using the paired non parametric test of Wilcoxon.

FIG. 10: GFAP expression in retinas from Harlequin and control eyes: RT-qPCR assays were performed with RNAs purified from 14 control retinas and from 9 pairs of retinas isolated from Hq mice in which one eye was subjected to intravitreal injection with AAV2/2-NGB (mice were euthanized between 5 to 6 months after vector administration). Relative GFAP mRNA variations were calculated using the comparative ΔΔCt method and the mitochondrial ATP6 gene as a normalizing gene. Values shown in the histogram are relative to the GFAP mRNA amount measured in control retinas and considered as 1; each value is the mean of all the triplicates obtained from each biological sample±S.E.M. Statistical significance was determined using the paired non parametric test of Wilcoxon. Primers used for the GFAP gene are shown in Table 7.

FIG. 11: Retinal ganglion cell evaluation in retinas from Harlequin eyes treated with AAV2/2-NGB vector: (A) RGC numbers were estimated in Hq treated and untreated eyes as well as in age-matched controls by immunolabeling for BRN3A and DAPI staining of retinal sections. BRN3A and DAPI-positive cells in the GCL were counted in 3-4 independent sections per eye for 24 control eyes and 13 Hq pairs of eyes in which only one was subjected to AAV2/2-NGB injection. Harlequin mice were euthanized between 5 to 6 months after vector administration and controls were aged about 7 months when euthanized. Histogram illustrates the results (mean values±S.E.M) corresponding to the overall RGC density (total number of BRN3A-positive cells per millimeter). Statistical significance was determined using the paired non parametric test of Wilcoxon. (B) RT-qPCR assays were performed with total RNAs extracted from control retinas and 14 pairs of retinas isolated from Hq mice in which one eye was subjected to intravitreal injection of AAV2/2-NGB (mice were euthanized between 5 to 6 months after vector administration). The steady-state levels of SNCG mRNA were compared to those obtained in RNA preparations from 18 age-matched control mouse retinas. Histogram shows the steady-state levels of SNCG mRNA calculated by the comparative ΔΔCt method and the mitochondrial ATP6 gene as a normalizing gene. Values shown are relative to the steady-state mRNA levels measured in control retinas and considered as 1; each value is the mean of all the triplicates obtained from each biological sample±S.E.M. Statistical significance was determined using the paired non parametric test of Wilcoxon.

FIG. 12: Morphological and functional evaluation of optic nerves from Harlequin mice after ocular AAV2/2-NGB treatment: Specific complex V (CV) and complex I (CI) enzymatic activities were assessed in single optic nerves isolated from 36 control mice, and from 24 Hq mice in which one eye was subjected to AAV2/2-NGB intravitreal injection (treated) and the contralateral one remained untreated. The successive measurements of CI and CV activities were expressed as nanomoles of oxidized NADH/min/mg protein. Histograms illustrate complex V activity (A) and complex I (B) as mean±S.E.M of each assay measured in triplicate. Values obtained in ONs from Hq mice subjected to vector administration, from untreated Hq mice and from age-matched controls were compared unpaired non parametric significance test of Mann-Whitney (*≦0.05, **≦0.01 and ***≦0.005); while data collected from Hq treated eyes and untreated controlateral eyes were compared using the paired non parametric significance test of Wilcoxon (*≦0.05, **≦0.01 and ***≦0.005).

FIG. 13: Preservation of nerve fibers in AAV2/2-NGB treated eyes protects Harlequin mouse vision: The Optomotry™ set-up allowed the determination of visual acuity threshold measurements (cycles per degree) for left and right eyes, independently scored (clockwise and couterclockwises responses) under photopic conditions. Visual acuities (right and left eye sensitivities) for Hq and control mice aged 4-8 weeks (n=7) are illustrated in (A) as means±S.E.M of measures performed twice 4-6 days apart. (B) Histogram shows visual acuities (right and left eye sensitivities) for 7 month-old control mice (n=22) and 18 Hq mice subjected to AAV2/2-NGB injection in their left eyes. Hq mice were evaluated 3 and 6 months post-injection (the test was performed twice each time 4-6 days apart), values represented are the means±S.E.M of measures performed 6 months after AAV2/2-NGB administration. Data collected from control and Hq were compared using the unpaired non parametric significance test of Mann-Whitney (*≦0.05, **≦0.01 and ***≦0.005). Data collected from Hq treated eyes and untreated contralateral eyes were compared using the paired non parametric significance test of Wilcoxon (*≦0.05, **≦0.01 and ***≦0.005).

### EXAMPLES

### Example 1

### 1.1 Material and Methods

### 1.1.1 Animals

Male Long Evans rats were used (Janvier, France). They were housed two per cage in a temperature-controlled environment, 12 h light/dark cycle. All animal studies were conducted in accordance with the guidelines issued by the French Ministry of Agriculture and the Veterinarian Department of Paris (Permit number DF/DF_2010_PA1000298), the French Ministry of Research (Approval number 5575) and the ethics committees of the University Paris 6 and INSERM (Authorization number 75-1710).

1.1.2 siRNA and shRNA Plasmid Construction

Anti-Ngb siRNA (5′ GUGAGUCCCUGCUCUACAU[dt]3′ SEQ ID NO: 22) or unspecific scrambled siRNA (5′GCCACACGAUUGCUGUCUU[dt]3′ SEQ ID NO: 23) were synthesized by Sigma-Aldrich. Rat RGCs were transfected with siRNAs (50 nM) and HiPerfect reagent (Qiagen, Valencia, Calif.) as recommended by the manufacturer. Anti-Ngb shRNA and anti-scrambled shRNA expression vectors targeting the same regions than the siRNAs were constructed in a GFP-expressing shRNA vector (pRNA-U6.1, Genscript, USA).

### 1.1.3 Purification of Retinal Ganglion Cells for Primary Cultures

Primary cell cultures were derived from 8 weeks-old rat retinas and purified by modifications of the sequential immunopanning described for young rats (B. A. Barres, B. E. et al, Neuron, 1 (1988) 791-803). RGCs were resuspended in half Neurobasal® medium (LifeTechnologies, Invitrogen) supplemented with B27 (1:50; LifeTechnologies, Invitrogen) and L-glutamine (2 mM; LifeTechnologies, Invitrogen) and half rat-retinal cell-conditioned culture medium (C. Fuchs, et al Invest Ophthalmol Vis Sci, 46 (2005) 2983-2991). RGC were seeded at 25,000 cells/cm² into 48-well tissue-culture plates containing glass-coverslips previously coated 1 h with poly-D-lysine (2 μg/cm²) and then with laminin 1 μg/cm² overnight (both from Sigma-Aldrich) (C. Fuchs, et al Invest Ophthalmol Vis Sci, 46 (2005) 2983-2991). siRNAs were added to the cells during the seeding in plates. Cells were incubated at 37° C. in a humidified atmosphere of 5% CO₂. Cells were counted with viability test at one and seven days or fixed before performing immunocytochemistry analyses. RGC viability was assessed with the “live-dead” test (LifeTechnologies, Invitrogen).

### 1.1.4 In Vivo Electroporation

The electroporation (ELP) procedure was performed in only one eye per rat essentially as described by Ishikawa and colleagues (H. Ishikawa, et al Gene Ther, 12 (2005) 289-298). Under anesthesia with isoflurane (40 mg/kg body weight), 20 μg of shRNA expression vectors were injected into the vitreous body, next the inventors proceeded to the ELP (S. Ellouze, et al, Am J Hum Genet, 83 (2008) 373-387). All the animals were euthanized 12 weeks after ELP.

1.1.5 Fundus Imaging by Confocal Scanning Laser Ophthalmoscopy (cSLO)

A digital cSLO (Heidelberg Engineering, Germany) with green laser illumination was used to examine nerve fiber layer (NFL) in each cardinal area of rat eyes before treatment and every three weeks until euthanasia. Pupil dilation was performed with topical 1% tropicamide (CibaVision, France). Rats were manually held in front of the apparatus, in an upright position. The built-in software was used for post-processing the images, including alignment, adjustment of contrast, construction of a composite image (M. Paques, et al Vision Res, 46 (2006) 1336-1345).

### 1.1.6 Optomotor Tests

The head-tracking method is based on an optomotor test devised by Cowey and Franzini in 1979 and frequently used since then in both rats and mice (A. Bouaita, et al, Brain, 135 (2012) 35-52). Long Evans rats were placed individually on an elevated horizontal platform surrounded by a motorized drum.

The protocol used yields independent measures of the acuities of right and left eyes based on the unequal sensitivities of the two eyes to pattern rotation: right and left eyes are most sensitive to counter-clockwise and clockwise rotations, respectively. A single blinded operator conducted all assessments, and codes were broken upon completion of data acquisition. The operator waited for the animal to settle in the chamber before initiating drum rotation. Vertical black-and-white lines of three varying widths, subtending 0.125, 0.25, and 0.5 cycles/degree (cyc/deg) were presented to the animal and rotated alternatively clockwise and counterclockwise, each for 60 s. This stimulated a subcortical reflex, so that a seeing animal involuntarily turned its head to track the moving lines. Head movements were recorded with a video camera mounted above the apparatus. Animals were scored only when the speed of the head turn corresponded to the speed of rotation of the stripes (12°/s). Light levels were kept constant (240 lux). Each animal was tested at four different time points by a single observer. During the experiments, a second masked grader re-evaluated the recorded videos to confirm the reliability of the scoring system.

### 1.1.7 Retinal and Optic Nerve Histology

Retinas or optic nerves were fixed in 4% PFA at 4° C., cryoprotected by overnight incubation in PBS containing 30% sucrose at 4° C. Retinas were embedded in OCT (Neg 50; Richard-Allan Scientific) and frozen in liquid nitrogen. Optic nerves were incubated in a 7.5% gelatin solution from porcine skin; Type A (Sigma-Aldrich) and 10% sucrose and frozen in 2-methyl-butane solution. Sections of retinas and optic nerves with a thickness of 10 μm were cut on a cryostat (Microm Microtech) and mounted on SuperFrost Plus slides.

TABLE 1
Antibody description

| Antibody | Type | Concentration | Supplier, reference |
|---|---|---|---|
| Ngb | Polyclonal | 5 μg/ml | Sigma, N-7162 |
| (histology) | | | |
| Ngb | Monoclonal | 5 μg/ml | Abcam, ab37258 |
| (histology) | | | |
| Ngb (western | Polyclonal | 1 μg/ml | Biovendor, RD181043050 |
| blot) | | | |
| BRN3A | Monoclonal | 1 μg/ml | Chemicon, MAB1585 |
| ATPsynthase | Monoclonal | 0.4 μg/ml | Invitrogen, LifeTechnologies, |
| β | | | A-21351 |
| ATPsynthase | Monoclonal | 0.4 μg/ml | Invitrogen, LifeTechnologies, |
| α | | | 459240 |
| TOMM 20 | Monoclonal | 0.4 μg/ml | Abcam, ab56783 |
| Alexa 488 | Rabbit | 4 μg/ml | Invitrogen, LifeTechnologies, |
| | | | A11008 |
| Alexa 594 | Mouse | 4 μg/ml | Invitrogen, LifeTechnologies, |
| | | | A11005 |

For immunochemistry, retinal sections were rinsed with PBS and treated with 1% BSA, 0.1% Triton and 0.05% Tween 20 in PBS for 1 h. They were then incubated with primary antibody overnight at 4° C. Sections were washed in PBS and incubated with the appropriate secondary antibodies and DAPI (2 μg/mL) for 2 h at room temperature. Primary and secondary antibodies used are shown in Table 1. Retinal flat mounts have been performed according to the protocol described by Paques and colleagues (M. Paques, et al Glia, 58 (2010) 1663-1668).

### 1.1.8 Microscopic Observations

Fluorescence labeling was monitored with: (i) a confocal laser scanning microscope (Olympus FV1000) Microscope control and image acquisition was conducted by using Olympus Fluoview® software version 3.1. (ii) Retinal sections were also scanned with the Hamamatsu Nanozoomer Digital Pathology (NDP) 2.0 HT, its Fluorescence Unit option (L11600-05) and the NanoZoomer's 3-CCD TDI camera (Hamamatsu Photonics, France). BRN3A-positive cells, as the estimation of overall RGCs, were assessed for each animal by manually counting three entire retinal sections as described earlier (A. Bouaita, et al, Brain, (2011)). A resolution of 0.23 μm per pixel (40×) was used routinely. Finally, all the images were analyzed with Photoshop and Image J.

1.1.9 RNA Extraction and qRT-PCR Assay

Total RNA from rat retinas were extracted using RNeasy Plus Mini kit from Qiagen. One microgram of total RNA was reverse-transcribed with oligo-dT using Superscript® II Reverse Transcriptase (LifeTechnologies, Invitrogen) following the manufacturer's instructions. NGB, Cytoglobin (CYGB), BRN3A, gamma-synuclein (SNCG) and ATP6 primers were customized to be specific for each mRNA species (Table 2) and synthesized by Invitrogen.

Quantitative PCR reactions were performed using ABI 7500 Fast (Applied Biosystems). The equivalent of 10 ng and 2 ng of cDNAs (relative to the whole RNA amount used for the reverse transcription) were used per gene as template for qPCR reactions with Power Sybr® green PCR Master Mix (Applied Biosystems) as recommended by the manufacturer. Each biological sample was subjected to the assay in triplicates per gene. Ct values were obtained by using ABI 7500 software (v.2.0.4) and the mitochondrial ATP6 gene was selected to normalize in order to obtain relative mRNA amount quantifications of each studied gene.

TABLE 2
Primers for qRT-PCR assays

| Gene | Primer Forward 5′-3′ | Primer reverse 5′-3′ |
|---|---|---|
| Ngb | CCAACGATGAAGGAGAGAGG | CAGAAATGCCGAACCAAGAG |
| | (SEQ ID NO: 24) | (SEQ ID NO: 25) |
| ATP6 | CAACCAACCTTCTAGGGCTTC | GCGGTAAGAAGTGGGCTAAA |
| | (SEQ ID NO: 26) | (SEQ ID NO: 27) |
| SNCG | GTAACCTCGGTGGCTGAGAA | TTCCAAGTCCTCCTTGCGTA |
| | (SEQ ID NO: 28) | (SEQ ID NO: 29) |
| BRN3A | AGGCCTATTTTGCCGTACAA | CGTCTCACACCCTCCTCAGT |
| | (SEQ ID NO: 30) | (SEQ ID NO: 31) |
| Cygb | GACTGACTTGCTCCGGAAAG | GTCTGAAGTGAGCGGGTGAG |
| | (SEQ ID NO: 32) | (SEQ ID NO: 33) |

### 1.1.10 Mitochondrial Purification and Western Blotting Analysis

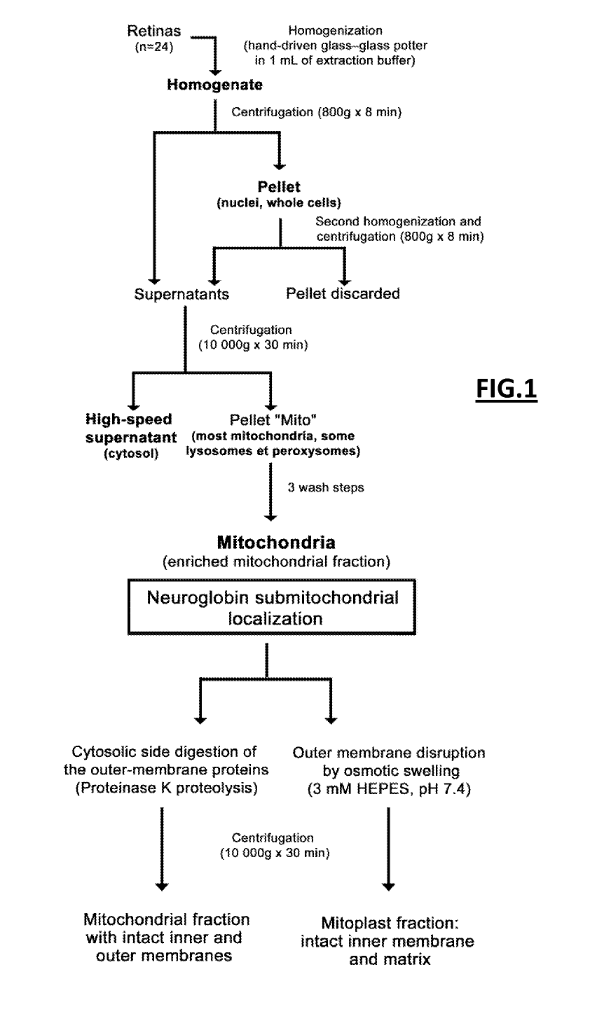

24 retinas were isolated from 8 week-old rats and washed in PBS at 4° C.; they were then homogenized in extraction buffer (0.32 M sucrose, 30 mM Tris-HCl; pH 7.6, 5 mM MgAc, 100 mM KCl, 0.1% fatty acid-free BSA, 5 mM β-mercaptoethanol, and 1 mM PMFS) and mitochondria were purified as previously described (V. Kaltimbache et alr, Rna, 12 (2006) 1408-1417) and as illustrated in FIG. 1. 30 μg of mitochondrial proteins were treated with 150 or 200 μg/mL proteinase K (PK) in the presence or absence of 1% Triton X-100 (v/v) at 4° C. for 30 min. The reaction was stopped by addition of 1 mM PMFS (Sigma-Aldrich). Samples were collected by centrifugation at 10000 g for 15 min at 4° C. For mitoplast preparation, aliquots of 30 μg of mitochondrial pellets (in 100 μL of extraction buffer) were subjected to osmotic shock by addition of 900 μL of 3 mM HEPES (pH 7.4) containing 1 mM of PMFS. After incubation on ice for 15 min, the suspension was centrifuged at 10000 g for 15 min to yield the mitoplast pellet. All the samples were then resolved in 12% or 15% SDS-PAGE and next transferred to a PVDF membrane. ?Membranes were probed with antibodies against Ngb, ATP synthase-α and TOMM20 (cf. Table 1). Immunoreactive bands were visualized with anti-mouse or anti-chicken coupled to horseradish peroxidase (0.1 mg/mL) followed by ECL Plus detection (Amersham International). Five independent mitochondria purifications were subjected to the analyses.

### 1.1.11 Tissue Homogenate Preparation and Respiratory Chain Enzymatic Assays

Optic nerves were prepared at 4° C. by homogenization of tissues using a 1 mL hand-driven glass-glass potter in 100 μL of extraction buffer (0.25 mM sucrose, 40 mM KCl, 2 mM EGTA, 1 mg/ml BSA, and 20 mM Tris-HCl, pH 7.2). Large cellular debris were spun down by a low speed centrifugation (1000 g×8 min) and supernatants were used immediately. Respiratory chain complex activities were measured using a Cary 50 spectrophotometer maintained at 37° C. (Varian, Australia) as previously described (P. Benit, et al, Clin Chim Acta, 374 (2006) 81-86). Each assay was made in duplicate with 20 μL of the homogenates obtained. Complex activity values were converted to specific activities after protein quantification by the Bradford method. All chemicals were of the highest grade from Sigma Chemical Company.

### 1.1.12 Statistical Analyses

Values are expressed as means±SEM (standard error of the mean).

Statistical analyses were performed with the GraphPad Prism5.0 software assuming a confidence interval of 95%. Data collected for all the independent observations were compared using the non parametric significance test of Mann-Whitney U (*≦0.05, **≦0.01 and ***≦0.005).

### 1.2 Results

### 1.2.1 Neuroglobin Profiles in Adult Rat Retinas

RGCs integrity is essential for visual function and their loss is directly involved in optic neuropathies and glaucoma. In an attempt to relate Ngb and mitochondria, the subcellular distribution of the mitochondrial protein ATP synthase-β (a subunit of the respiratory chain complex V) and the Ngb protein by immunostaining of retinal sections have been compared. Consistent with other reports, cells positive for antibodies against Ngb were found in the photoreceptor layer (PL), the inner nuclear layer (INL) and the GCL (Data not shown). It clearly appeared that Ngb is highly expressed in RGCs, specifically labeled with an antibody against the transcription factor BRN3A. To further support the presence of Ngb in RGCs and its possible localization to mitochondria, retinal flat mounts and optic nerve sections from adult rats were examined and processed for immunohistochemistry using anti-Ngb associated to anti ATP synthase-β (Data not shown). Immunolabeled RGCs showed a cytoplasmic, a dendritic, and an axonal distribution of Ngb protein. The immunoreactivity of the Ngb antibody was often revealed as a punctuate distribution of fluorescent dots excluded from the nuclei, similar to the signal obtained with anti-ATP synthase-3 antibody; both antibodies revealed an elevated extent of colocalization (Data not shown). Moreover, results clearly show a homogenous signal of Ngb in RGC somas and their dendrites in the IPL as well as in their axons in the Nerve Fiber Layer (NFL). Longitudinal optic nerve sections from adult rats were also performed and processed for immunohistochemistry using anti-Ngb associated with anti-ATP synthase-β. Ngb signals observed in optic nerves not only overlapped with ATP synthase-β signals but also confirmed the presence of mitochondrial labeling of RGC axons which can be distinguished from that of the resident optic nerve cells (Data not shown). Consequently, Ngb subcellular localization matches to some extent with the pattern of mitochondrial network revealed by ATP synthase-β in both RGC bodies and their axons.

### 1.2.2 Neuroglobin Subcellular Localization in Retinal Neurons

To ascertain whether Ngb can be detected inside the mitochondria, organelle fractions purified by differential centrifugation (FIG. 1) from rat retinas were subjected to immunoblot analyses. Results show that specific antibodies against Ngb recognized three proteins of ˜17, ˜19 and ˜21 kDa apparent molecular mass, in enriched mitochondria fractions; only the 17 kDa form was found in the cytosol. To compare the proportion of Ngb in the cytosol or the mitochondria relative to the total amount of Ngb in homogenates, four independent experiments of subcellular fractionation (as described in FIG. 1) were compared. The results indicate that: (i) Signals obtained with antibody against Ngb or ATP synthase a were enriched in mitochondrial fractions relative to whole homogenates or cytosols; (ii) when the sum of the Ngb signals in mitochondrial and cytosolic factions were calculated it appears that approximately 70% of the overall Ngb signal was revealed in the mitochondrial compartment.

To further evaluate if Ngb was completely translocated inside the organelle, mitochondria fractions were treated with PK; significant amounts of the three forms of Ngb and ATP synthase-α are insensitive to PK-induced proteolysis, thus indicating that Ngb forms were truly integrated into the mitochondria and hence remain detectable on immunoblotting (Data not shown). Next, mitochondrial fractions were treated with PK and Triton X-100; theoretically the detergent disrupts both mitochondrial membranes and leads to the entire proteolysis of mitochondrial proteins demonstrating their localization inside the organelle in a protease-sensitive form. Results clearly show that ATP synthase-α and Ngb protein signals became diminished as they were digested by PK in the presence of detergent.

To determine whether Ngb could be present either in the intermembrane space or the internal side of the inner membrane; mitoplasts were obtained by subjecting the samples to osmotic shock. The outer membrane protein TOMM20 was significantly digested by PK, as expected since a portion of it is exposed to the cytosolic side, moreover the signal entirely disappeared in mitoplasts. By ?contrast, the three forms of Ngb were mostly preserved as is ATP synthase-α (Data not shown). Hence, the three forms of Ngb identified by Western blotting of mitochondrial fractions were fully integrated inside the organelle and they could be further contained in the matrix or in the inner membrane.

### 1.2.3 Neuroglobin Knockdown in Rat Purified Primary Culture of Retinal Ganglion Cells by Small Interfering RNA

The inventors examined whether the inhibition of NGB expression influenced RGC survival and neurite outgrowth in primary RGC cultures via the small interfering RNA (siRNA) strategy.

To study the impact of NGB knockdown on cell survival and neurite outgrowth, the anti-Ngb siRNA or the scrambled siRNA were associated with transfection agent and added on the 1^{st} day of RGC cultures in rat retinal cell-conditioned medium. Under this culture condition RGCs survive 7-12 days and developed neuritic processes that may extend for several cell-body diameters and were often branched (Data not shown). After seven days of culture, many viable cells develop neuritic processes and immunoreactivity obtained with the antibody against Ngb was similar to that observed with the antibody against the mitochondrial ATP synthase-β polypeptide, as previously observed in vivo (see point 1.2.1). Indeed, both proteins were distributed along the neuritic processes and in the cytosol of RGC primary culture in control conditions (Data not shown). Furthermore, it clearly appears that Ngb immunostaining was strongly reduced (Data not shown) compared to cells transfected with scrambled siRNA or untreated cells.

Cell survival was assessed by counting live RGCs from dead-cell population. In controls, an approximate 50% diminution in the total amount of living cells was observed after 7 days of culture, an expected phenomenon related to the vulnerability of these adult neurons once their axons are disrupted. In fact, previous reports described that after 10 days of culture, the cell density was reduced to 10% in control conditions and to 30% when conditioned medium obtained from retinal cells was used. Despite this effect, the inventors observed a significant and more pronounced decrease (˜3-fold) in cell survival when RGCs were transfected with anti-Ngb siRNA (Table 3) relative to either control conditions or scrambled siRNA treatment. The differences were significant according to the Mann-Whitney non parametric test (p: 0.0007 or 0.02 respectively). This result indicates that NGB expression is essential for RGC survival in vitro.

TABLE 3
Cell survival was estimated using the “live-dead” test. Only alive
RGCs were counted from ten fields selected
identically on each coverslip of three
independent RGC cultures at one or seven days. Results
were presented as cell survival relative to control
conditions (mean values ± S.E.M, standard error for the
mean).

| | Day 1 (number of | Day 7 (number of |
|---|---|---|
| Total number of RGCs | independent counts) | independent counts) |
| Control | 85 ± 35 (7) | 46.6 ± 18.14 (7) |
| Anti-Ngb siRNA treatment | 92 ± 20 (6) | 16.0 ± 4.0 (10) |
| P value Ngb/Control | 1 | 0.007 |
| P value Ngb/scrambled | 1 | 0.02 |
| Scrambled siRNA | 96 ± 38 (4) | 29.3 ± 9.7 (7) |
| treatment | | |
| P value Scrambled/Control | 0.48 | 0.08 |

### 1.2.4 In Vivo Knockdown of Neuroglobin Expression in Retinal Ganglion Cells of Adult Rats and its Impact on Nerve Fiber Density

The impact of NGB expression attenuation was assessed in vivo by ELP (S. Ellouze et al, Am J Hum Genet, 83 (2008) 373-387), after injection in the vitreous body of one eye of a plasmid DNA leading to the synthesis of a short hairpin RNA (shRNA) anti-Ngb. The in vivo ELP procedure results in a highly efficient gene delivery to the GCL since more than 50% of RGCs express the transgene for at least 2 months. NFL integrity was evaluated using confocal Scanning Laser Ophthalmoscopy (cSLO), which represents a powerful technique for in vivo imaging of rodents eye fundus (A. Bouaita, et al, Brain, 135 (2012) 35-52). Striations of NFL radiating from the optic disc were clearly visible in each eye from rats subjected to ELP. Each area of the eye fundus was visualized before and at different times after treatment until euthanasia. The inventors analyzed nerve fiber density before and 3 months after scrambled shRNA plasmid administration: eye fundus visualizations did not show any darker or thinner striations, when compared to the ones obtained before ELP or to untreated eyes. In contrast, a noticeable loss of nerve fiber bundles was evidenced in eyes electroporated with anti-Ngb shRNA (Data not shown). The NFL striation loss, especially in the superior and inferior retinal areas, reflects RGC axon degeneration and was noticed one month after the ELP. Nerve fiber disappearance was observed in ˜25% of treated anti-Ngb shRNA eyes (n=24); while eye fundus from all the eyes electroporated with the scrambled shRNA plasmid (n=22) or untreated eyes (n=46) showed well preserved tracks of axons in all the areas visualized until euthanasia (Data not shown). These results indicated that RGC axons in the NFL of eyes treated with anti-Ngb shRNA have undergone a degenerative process, confirming the deleterious effect of in vivo NGB knockdown.

1.2.5 Relative Abundance of Neuroglobin and Specific Retinal Ganglion Cells mRNAs

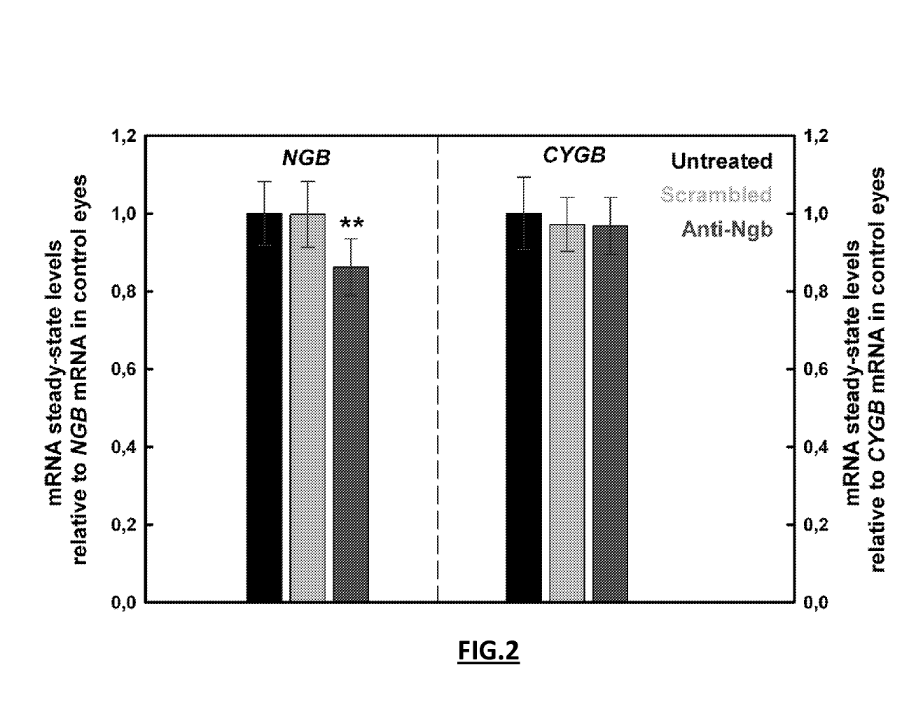

To substantiate that the anti-Ngb shRNA treatment was efficient in reducing NGB mRNA levels, its abundance in retinas was determined by quantitative real-time PCR of reverse-transcribed mRNAs (RT-qPCR) using the comparative ΔΔCt method and the mitochondrial ATP6 gene as “normalizing” gene. RNA preparations from retinas of 18 control eyes, 8 eyes treated with scrambled shRNA or 10 eyes treated with anti-Ngb shRNA, from rats euthanized 3 months after ELP, were examined. The relative amount of NGB mRNA in anti-Ngb shRNA treated retinas was 13.8% less abundant than in retinas isolated from the 18 untreated eyes; this difference was significant according to the Mann-Whitney test (p=0.023) (FIG. 2). The decrease of NGB mRNA may appear small, but RNAs were prepared from the whole retinal cell population. Thus, NGB mRNA levels of RGCs transduced with the anti-Ngb shRNA cannot be discriminated from those of untransduced cells which also expressed NGB (bipolar and photoreceptor; cf. point 1.2.1), whose proportion is very high relative to RGCs; indeed, the fraction of RGCs relative to the total cell population was estimated at less than 1% in adult mouse retina (C. J. Jeon, et al, J Neurosci, 18 (1998) 8936-8946). The administration of the plasmid directing the synthesis of scrambled shRNA did not change the relative abundance of NGB mRNA in treated retinas compared to retinas from untreated eyes (p=0.51) (FIG. 2). Additionally, the relative amount of CYGB mRNA encoding another hexacoordinated globin was evaluated (C. Lechauve, et al, Febs J, 277 (2010) 2696-2704); it did not change in any of the retinas examined (p=0.87 and 0.22 for scrambled and anti-Ngb shRNA respectively) confirming that anti-Ngb shRNA plasmid administration leads specifically to the diminution of NGB mRNA steady-state levels (FIG. 2).

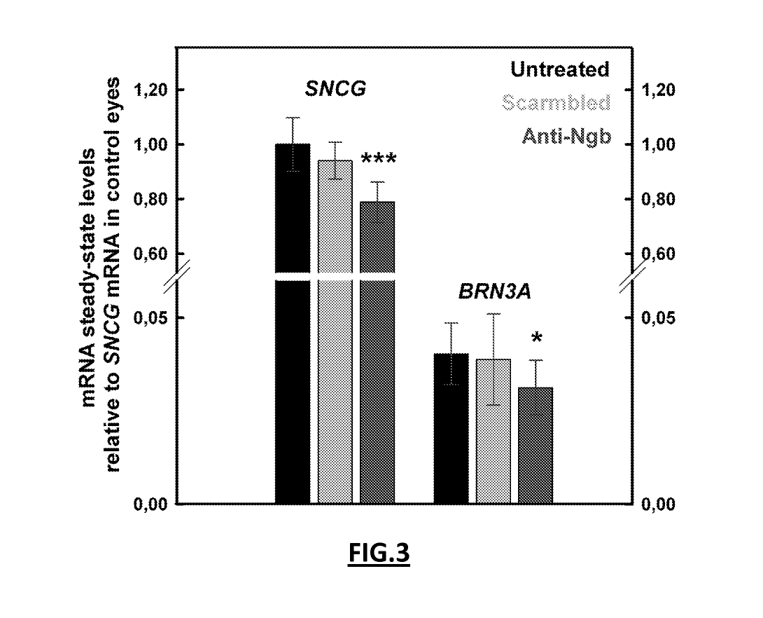

The amount of SNCG and BRN3A mRNAs were also measured since they are highly abundant in adult RGCs, while they are almost undetectable in other retinal neurons. Anti-Ngb shRNA-treated eyes showed a ˜20% reduction of both SNCG and BRN3A mRNA levels relative to values measured in retinas from control eyes (p=0.002 and 0.033 for SNCG and BRN3A respectively) (FIG. 3); Whereas, steady-state levels of SNCG and BRN3A mRNAs did not change in eyes electroporated with the scrambled shRNA relative to controls: p=0.17 and 0.95 for SNCG and BRN3A respectively (FIG. 3). Thus, NGB knockdown was effective on reducing NGB mRNA level which results in a ˜20% reduction of SNCG and BRN3A mRNA amounts. This diminution could reflect RGC loss, since SNCG or BRN3A expression is considered as an index of RGC number (R. Torero Ibad, et al J Neurosci, 31 (2011) 5495-5503).

### 1.2.6 Deleterious Effect of Neuroglobin Knockdown on Retinal Ganglion Cells Integrity In Vivo

To corroborate the negative impact of NGB knockdown on RGC integrity, retinal sections from rats euthanized 3 months after the treatment were examined by immunochemistry using antibodies against Ngb and BRN3A proteins. Retinal sections of anti-Ngb shRNA treated animals presenting a noticeably loss of nerve fibers (Data not shown) showed an important diminution of BRN3A-positive cells and the Ngb immunostaining signal in the GCL relative to the signals observed in the accompanying untreated eye (Data not shown, rat #1); an additional rat retinal section is shown in which a more subtle reduction of BRN3A-positive cells was evidenced (Data not shown rat #5). Ngb staining in the other retinal layers was similar in treated and control eyes (Data not shown rat #1 and #5). On the other hand, no evident changes in BRN3A-positive cells were noticed in eyes electroporated with scrambled shRNA (Data not shown, rat #4). Cryostat sections of retinas were counted for BRN3A-positive cells in the GCL to estimate the number of RGCs in 8 eyes electroporated with anti-Ngb shRNA, 7 eyes electroporated with scrambled shRNA and 10 untreated eyes.

TABLE 4
RGC densities were calculated after immunolabeling
for BRN3A antibody and DAPI staining; this later
allowing estimation of total nuclei in the GCL.
BRN3A and DAPI-positive cells in the GCL
were counted in three independent retinal
sections per animal: 10 control eyes, 7 eyes treated
with scrambled shRNA and 8
eyes treated with anti-Ngb shRNA. Results were
presented as cell density/mm relative
to control conditions (mean values ± S.E.M, standard
error for the mean, student t test).

| | Cell density per mm | |
|---|---|---|
| | Total cells of GCL | RGCs |
| Control eyes (n = 10) | 121.1 ± 4.2 | 48.4 ± 3.0 |
| Anti-Ngb shRNA treated eyes | 116.7 ± 8.7 | 40.1 ± 4.3 |
| (n = 8) | | |
| P value Ngb/control | 0.60 | 0.001 |
| P value Ngb/scrambled | 0.60 | 0.005 |
| Scrambled shRNA treated | 120.9 ± 4.6 | 47.1 ± 3.0 |
| eyes (n = 6) | | |
| P value scrambled/control | 1 | 0.25 |

The inventors found that RGC density (overall number of RGCs/mm) was reduced in anti-Ngb shRNA-treated retinas relative to control retinas (Table 4), the diminution of about 20% was significant according to the Mann-Whitney t test: p=0.001. Conversely, RGC density in eyes treated with scrambled shRNA was not significantly different to controls: p=0.25 (Table 4). Hence, in vivo ELP with anti-Ngb shRNA leads to a significant RGC loss which supports the optic fiber disappearance evidenced by eye fundus imaging (Data not shown).

### 1.2.7 Impact of Neuroglobin Knockdown on Respiratory Chain Activity in Optic Nerves

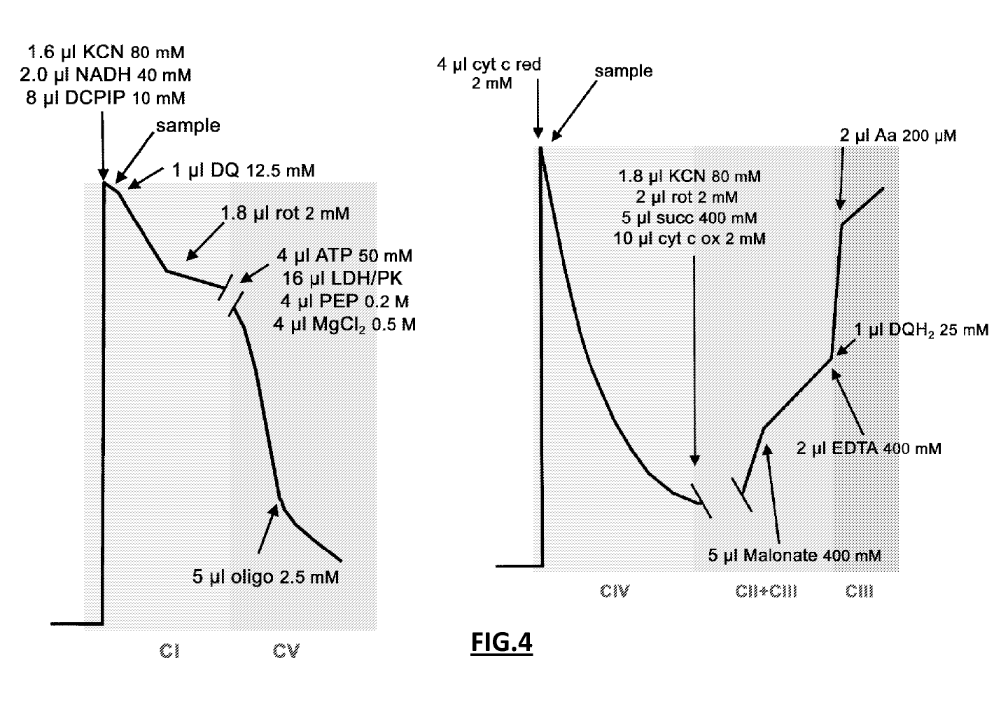

Since the number of BRN3A-positive cells and eye fundus imaging indicated that the knockdown of NGB expression leads to RGC degeneration, the inventors evaluated whether respiratory chain function can be hampered in optic nerves isolated from animals sacrificed 3 months after anti-Ngb shRNA treatment. The spectrophotometric method used for assessing enzymatic activities of respiratory chain complexes has been successfully applied to accurately detect isolated defects in small amounts of tissue homogenates (P. Benit, et al, Clin Chim Acta, 374 (2006) 81-86). Two independent spectrophotometric assays were devised to sequentially measure in homogenates of single optic nerves the enzymatic activities of: (1) rotenone-sensitive complex I (CI) and oligomycin-sensitive complex V (CV); (2) complex IV (CIV), malonate-sensitive combined complex II+III (CII+CIII) and antimycin-sensitive complex III (CIII) (FIG. 4). A 30% decrease relative to control values was observed for CI and CIII specific activities in optic nerves of eyes electroporated with anti-Ngb shRNA (n=17) relative to control optic nerves (n=32); the differences were significant according to the Mann-Whitney test; p values were 0.001 and 0.051 for CI and CIII activities respectively (Table 5). Conversely, CI and CIII activities in optic nerves from eyes electroporated with scrambled shRNA (n=15) were not significantly different from those measured in untreated eyes (p values: 0.64 and 0.63 respectively). Besides, CII, CIV and CV activities were similar between anti-shRNA treated eyes and controls (Table 5). Thus, NGB knockdown in retinas interferes in a specific manner on respiratory chain function in optic nerves, since the defect was specifically demonstrated in CI and CIII enzymatic activities.

TABLE 5
Specific activities assessed in optic nerves from 32 control eyes, 17 eyes
treated with anti-Ngb shRNA or 15 treated with scrambled shRNA are shown.
Abbreviations: CI-CV, various complexes of the respiratory chain; Aa, antimycin A; ATP,
adenosine triphosphate; Cyt, cytochrome; DCPIP, dichlorophenol indophenol; DQ, DQH2,
duroquinone (oxidized), duroquinol (reduced), respectively; EDTA, ethylene diamine
tetraacetic acid; KCN, potassium cyanide; LDH, lactate dehydrogenase; MgCl2,
magnesium chloride; NADH, reduced nicotinamide adenine dinucleotide; oligo,
oligomycin; ox, oxidized; PEP, phosphoenol pyruvate; PK, pyruvate kinase; red, reduced;
rot, rotenone; succ, succinate.

| Origin of optic nerves | Specific activities (nmol/min/mg protein) ± S.E.M | | | | |
|---|---|---|---|---|---|
| and number tested (n) | CI | CII + CIII | CIII | CIV | CV |
| Control eyes (n = 32) | 68.3 ± 14.3 | 19.4 ± 6.1 | 82.7 ± 26.4 | 102.2 ± 32.5 | 115.3 ± 45.3 |
| Scrambled shRNA | 65.8 ± 11.0 | 19.2 ± 3.6 | 88.3 ± 25.6 | 102.8 ± 21.5 | 120.3 ± 38.5 |
| treated eyes (n = 15) | | | | | |
| Mann-Whitney test | 0.64 | 0.77 | 0.63 | 0.86 | 0.74 |
| Anti-Ngb shRNA treated | 48.8 ± 15.8 | 16.9 ± 7.3 | 64.7 ± 22.7 | 88.0 ± 38.3 | 99.3 ± 46.4 |
| eyes (n = 17) | | | | | |
| Mann-Whitney test | 0.001 | 0.34 | 0.051 | 0.26 | 0.40 |
| (/control) | | | | | |
| Mann-Whitney test | 0.016 | 0.46 | 0.043 | 0.30 | 0.40 |
| (/scrambled) | | | | | |

1.2.8 Functional Assessment of Visual Responses in Animals Treated with Anti-Ngb shRNA

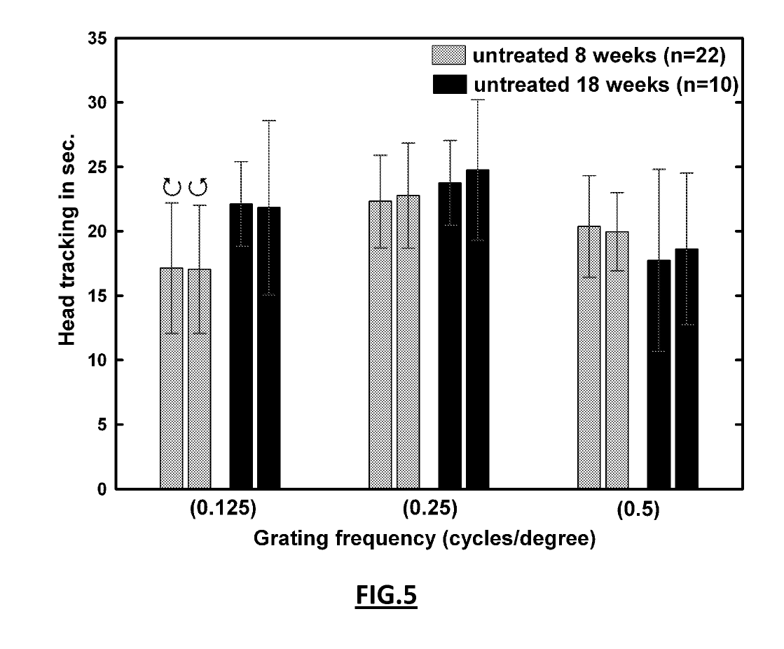

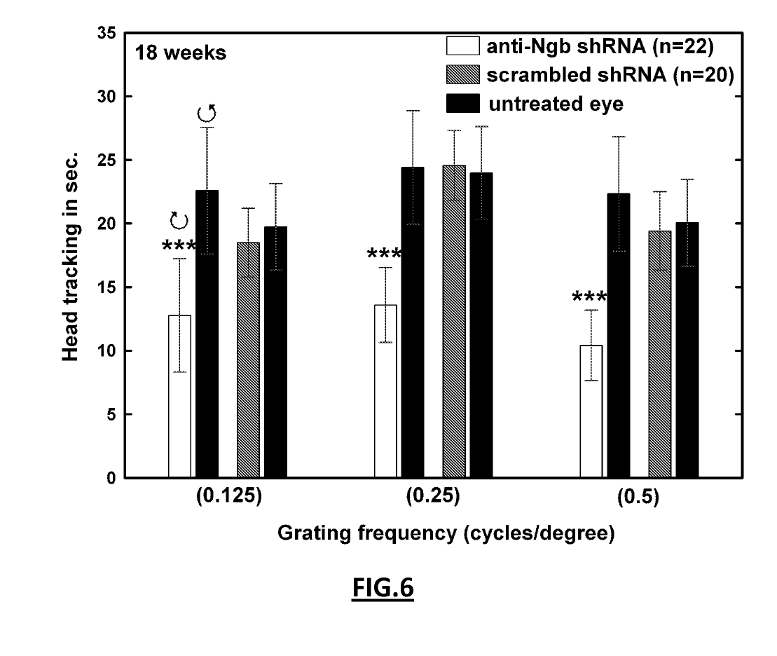

The functional consequences of RGC loss due to anti-Ngb shRNA treatment on visual function was assessed by optomotor head-tracking experiments (R. M. Douglas, et al, Vis Neurosci, 22 (2005) 677-684). Tracking capability was examined in both clockwise and counter clockwise drum rotations at three frequencies: 0.5, 0.25 and 0.125 cycles per degree. Because only temporal-to-nasal motion is effective through each eye, clockwise movement will drive tracking through the left eye, whereas counterclockwise motion will activate the right eye. FIG. 5 shows visual responses assessed in 8 and 18 week-old control rats (n=22 and n=10); they mostly presented head tracking scores of similar magnitude for the clockwise or counterclockwise drum rotations. FIG. 6 shows data collected from rats 10 weeks after the treatment with scrambled shRNA or anti-Ngb shRNA plasmid. Rats treated with scrambled shRNA (n=20) showed no significant difference between clockwise and counterclockwise responses for all the three spatial frequencies tested (0.125 p=0.30 at 0.125 cycles per degree, p=0.94 at 0.25, and p=0.68 at 0.5. Moreover, their clockwise responses before plasmid administration and 10 weeks later were very similar for instance for the 0.5 cycle per degree frequency the p value calculated was 0.41 (FIG. 5, untreated 8 week-old and FIG. 6 scrambled shRNA). Conversely, an unequivocal decline in visual performance in rats treated with anti-Ngb shRNA was measured (n=22) which responded with poor clockwise scores; indeed they spent much less time tracking across the test period for the clockwise drum rotation relative to the counterclockwise responses. The decline in visual performance was statistically different (p<0.0001 at all three frequencies); which represents a 46% decrease of that measured for their accompanying control eyes (FIGS. 5 and 6). Obviously when the clockwise responses of these animals were compared to the one collected from untreated animals of 18 week-old (FIG. 5, untreated 18 week-old) the diminution of about 43% is also significant (p<0.005 at all three frequencies). Thus, RGC loss due to NGB knockdown has a negative impact on head-tracking behavior; indicating the direct connection between NGB expression, respiratory chain integrity and visual function.

### Example 2

### 2.1 Material and Methods

### 2.1.1 Animals and Diets

The Hq strain was B6CBACaAw-J/A-Pdc8Hq/J obtained from Jackson Laboratory (http://jaxmice.jax.org/strain/000501.html). These mice exhibit the main features of human neurodegenerative diseases due to respiratory chain complex I (RCCI) deficiency, such as the degeneration of the cerebellum, retina, optic nerve, thalamic, striatal, and cortical regions. This complex phenotype is caused by the knockdown of the nuclear gene AIF encoding the mitochondrial Apoptosis Inducing Factor, which levels drops to less than 10% of the amount seen in wild-type mice (Klein J A, et al. (2002) Nature 419: 367-374), and leads to RCCI deficiency (Vahsen N, et al. (2004) Embo J 23: 4679-4689). All Hemizygous (Hq/Y) males used in this study were F1 mice bred from founders having a mixed genetic background. Hemizygous (Hq/Y) males were the recipient of evaluations and gene therapy; they were compared exclusively to the littermate males from the colony. The mice were housed from one to four per cage in a temperature-controlled environment, 12-h light/dark cycle and free access to food and water. Studies were conducted in accordance with the statements on the care and use of animals in research of the guidelines issued by the French Ministry of Agriculture and the Veterinarian Department of Paris (Permit number DF/DF_2010_PA1000298), the French Ministry of Research (Approval number 5575) and the ethics committees of the University Paris 6 and the INSERM, Institut National de la Sante et de la Recherche Médicale (Authorization number 751710).

### 2.1.2 Adeno-Associated Viral Vector and Intravitreal Injections

The Mus musculus Neuroglobin (NGB) mRNA sequence of 1630 base pairs (bp) (NM_022414.2, SEQ ID NO: 5) was synthesized by Genscript Corp (Piscataway, N.J. 08854 USA). It encompasses the full-length 5′UTR (279 bp, SEQ ID NO: 6), the entire Open Reading Frame (ORF; SEQ ID NO: 8) encoding 151 amino acid-long protein, and two restriction sites for cloning into the pAAVIRES-hrGFP vector. The hGH (human growth hormone 1) polyadenylation signal was replaced by the full-length 3′UTR of NGB (895 bp, SEQ ID NO: 7). NGB transcription is under the control of the Cytomegalovirus promoter and the β-globin intron for ensuring high levels of expression. The ORF is in frame with the 3× FLAG® sequence at the C-terminus. The pAAV-IRES-hrGFP vector (http://www.genomics.agilent.com/) has a dicistronic expression cassette in which the humanized recombinant green fluorescent protein (hrGFP) is expressed as a second ORF translated from the encephalomyocarditis virus internal ribosome entry site (IRES). The final vector, named AAV2/2-NGB (SEQ ID NO: 9), contains AAV2 inverted terminal repeats (ITRs), which direct viral replication and packaging. The expression cassettes flanked by the two AAV2 ITRs, were encapsidated into AAV2 shells. Vectors were produced by the “Centre de Production de Vecteurs and the INSERM UMR1089, Nantes” (http://www.atlantic-genetherapies.fr/). The rAAV titers were determined by dot blot and expressed as vector genomes (VG) per mL; 1×10¹² VG/mL. For intravitreal injections, after dilatation of the pupil with topical 1% tropicamide (CibaVision, France), mice were subjected to anesthesia with isoflurane (40 mg/kg body weight). The tip of a 33-gauge needle, mounted on a 10 μl Hamilton syringe (Hamilton Bonaduz AG, Bonaduz, Switzerland) was advanced through the sclera and 2 μL of vector suspension (2×10⁹ VG) was injected intravitreally, avoiding retinal structure disruption, bleeding or lens injury. Viral particles were extemporaneously mixed with 1/10000 of fluorescein to follow the homogenous dissemination of the suspension into the vitreous body. Forty five mice were subjected to AAV2/2-NGB administration during the course of this study. One animal suffered from a haemorrhage after intravitreal injection; it was euthanized and discarded from the study. Further, two mice died few weeks after the treatment due to technical errors in the animal facility and one died from natural causes.

### 2.1.3 Fundus Imaging by Confocal Scanning Laser Ophthalmoscopy

A digital confocal Scanning Laser Ophthalmoscope, cSLO (Heidelberg Engineering, Germany) was used to examine nerve fiber layer (NFL) in each cardinal area of mouse eyes before treatment and different times after vector administration as previously described (Paques M, et al. (2006) Vision Res 46: 1336-1345). Briefly, all examinations were carried out in manually restrained conscious animals which were held in front of the cSLO objective after pupil dilation; the overall duration of each examination was 1 minute per eye. Stacks of 30 images (1,500 μm of approximate width and a definition of 512×512 pixels) were acquired at different planes of focus to capture the whole surface of the retina.

### 2.1.4 Optomotor Response

Visual acuity was measured, under photopic conditions, by observing the optomotor responses of mice to rotating sinusoidal gratings (OptoMotry™) (Prusky G T, et al (2004) Invest Ophthalmol Vis Sci 45: 4611-4616). Mice reflexively respond to rotating vertical gratings by moving their head in the direction of grating rotation. The protocol yields independent measures of right and left eye acuities based on the unequal sensitivities of the two eyes to pattern rotation: right (untreated) and left (treated) eyes are most sensitive to counterclockwise and clockwise rotations, respectively (Prusky G T, et al (2004) Invest Ophthalmol Vis Sci 45: 4611-4616). Each mouse was placed on a pedestal located in the centre of four inward facing LCD computer monitors screens. Once the mouse became accustomed to the pedestal, the test was initiated by presenting the mouse with a sinusoidal striped pattern that rotates either clockwise or counter-clockwise and varying widths. Spatial frequency of the grating was randomly increased by the software until the animal no longer responded. As the mouse moved about the platform, the experimenter followed the mouse's head with a crosshair superimposed on the video image. When a grating perceptible to the mouse was projected on the cylinder wall and the cylinder was rotated (12°/s), the mouse would typically start to track the grating with reflexive head movements in concert with the rotation. The short testing reduced the possibility of adapting to the stimulus and established that each animal was capable of tracking when a changed stimulus was present. The process of changing the spatial frequency of the test grating was repeated a few times until the highest spatial frequency the mouse could track was identified as the threshold which defined the visual acuity. Experimenters were masked to the treatment and to the animal's previously recorded thresholds.

### 2.1.5 Retinal and Optic Nerve Histology

Retinas and optic nerves (ONs) were carefully collected and fixed in 4% PFA at 4° C., cryoprotected by overnight incubation in PBS containing 30% sucrose at 4° C. Retinas were embedded in OCT (Neg 50; Richard-Allan Scientific), frozen in liquid nitrogen and optic nerves were embedded in a solution of PBS+7.5% gelatin from porcine skin Type A (Sigma-Aldrich) and 10% sucrose and frozen in a 2-methyl-butane solution at −45° C. Sections of retinas and ONs were cut (10 μm thickness) on a cryostat (Microm HM560, Thermo Scientific) at −20° C. and mounted on SuperFrost®Plus slides.

For immunochemistry, sections of retinas and ONs were permeabilized with 0.1% Triton X-100 in PBS for 15 minutes at room temperature and treated with 3% BSA, 0.1% Triton and 0.05% Tween 20 in PBS for 1 hour. They were then incubated with primary antibody overnight at 4° C. The next day, sections were washed three times in PBS and incubated with the appropriate secondary antibodies and 2 μg/mL of 4′,6-diamidino-2-phenylindole (DAPI) for 2 hours at room temperature with 3% BSA, 0.1% Triton and 0.05% Tween 20 in PBS. At last, they were washed 3 times with PBS, rinsed with sterile water and mounted on a glass slide. Primary and secondary antibodies used are shown in table 6.

TABLE 6
Antibody description

| Antibody | Type | Concentration | Supplier, reference |
|---|---|---|---|
| NGB (histology) | Polyclonal | 5 μg/mL | Sigma, N-7162 |
| NGB (western blot) | Polyclonal | 1 μg/mL | Biovendor, RD181043050 |
| AIF (western blot) | Monoclonal | 0.4 μg/mL | Millipore, AB16501 |
| ATP synthase β | Monoclonal | 0.2 μg/mL | Invitrogen, LifeTechnologies, A-21351 |
| (western blot) | | | |
| GFP (histology) | Polyclonal | 20 μg/mL | Abcam, ab56783 |
| NDUFB6 (histology) | monoclonal | 10 μg/mL | Invitrogen, LifeTechnologies, A21359 |
| BRN3A (histology) | Monoclonal | 1 μg/mL | Chemicon, MAB1585 |
| GFAP (histology) | Polyclonal | 2.9 μg/mL | Sigma Aldrich, G3893 |
| NF200 (histology) | Monoclonal | 1 μg/mL | Chemicon, MAB1585 |
| FLAG (histology) | Monoclonal | 5 μg/mL | Sigma Aldrich, F1804 |
| Alexa 488 | Rabbit | 4 μg/mL | Invitrogen, LifeTechnologies, A11008 |
| Alexa 594 | Mouse | 4 μg/mL | Invitrogen, LifeTechnologies, A11005 |

Immunofluorescence analyses with antibodies against BRN3A and GFP were performed on retinal sections from Hq mice in which one eye was subjected to intravitreal administration of the AAV2/2-NGB vector while the contralateral eye remained untreated. Mice were sacrificed about 22 weeks after vector administration. Retina from the untreated eye displayed very weak GFP staining and few BRN3A-positive cells in the GCL. Conversely, retinal section from AAV2/2-NGB treated eye showed strong GFP and BRN3A labeling, noticeable throughout the GCL; moreover, GFP staining is also noted in the NFL. Noteworthy the overall number of BRN3A-positive cells was higher in treated retinas than in untreated ones.

### 2.1.6 Microscopic Observations

Fluorescence labeling was monitored in the Cellular Imaging Facility of the Institute with: (i) a confocal laser scanning microscope (Olympus FV1000) Microscope, image acquisition was conducted by using Olympus Fluoview® software version 3.1. (ii) Retinal sections were also scanned with the Hamamatsu Nanozoomer Digital Pathology (NDP) 2.0 HT, its Fluorescence Unit option (L11600-05) and the NanoZoomer's 3-CCD TDI camera (Hamamatsu Photonics, France). BRN3A-positive cells, as the estimation of overall RGCs, were assessed for each animal by manually counting 2-4 entire retinal sections as described earlier (cf. example 1). Lastly, all the images were analyzed with Photoshop and Image J.

### 2.1.7 RNA Extraction and RT-qPCR Assay

Total RNA from rat retinas were extracted using RNeasy Plus Mini kit from Qiagen. To ensure the absence of DNA a treatment with RNase-free DNase (Qiagen) and a subsequent cleanup with the RNeasy MinElute cleanup kit (Qiagen) were performed. This was confirmed by subjecting 10 ng of each RNA preparation to qPCR with specific primers for the NGB transgene and the mitochondrial ATP6 gene. One micrograms of total RNA was reverse transcribed with oligo-dT using Superscript® II Reverse Transcriptase (Life Technologies). Quantitative PCR reactions were performed using ABI 7500 Fast (Applied Biosystems) and the specific primers listed on Table 7.

TABLE 7
Primers for RT-qPCR assays

| Gene | Primer Forward 5′-3′ | Primer reverse 5′-3′ |
|---|---|---|
| NGB | CTCAGGCAAGGGAAGCATAG | CAGTTAGGTTTCCCCCAAAA |
| | (SEQ ID NO: 10) | (SEQ ID NO: 11) |
| NGB-AAV | AGGCTATGTCACGAGGTTGG | GGGTAACCCTATGCAGTCGT |
| | (SEQ ID NO: 12) | (SEQ ID NO: 13) |
| ATP6 | CGTAATTACAGGCTTCCGACA | AGCTGTAAGCCGGACTGCTA |
| | (SEQ ID NO: 14) | (SEQ ID NO: 15) |
| SNCG | GGAGGCAGCTGAGAAGACC | ACTGTGTTGACGCTGCTGAC |
| | (SEQ ID NO: 16) | (SEQ ID NO: 17) |
| GFAP | CCCGTTCTCTGGAAGACACT | CTTCAGGGCTGAGAGCAGTC |
| | (SEQ ID NO: 18) | (SEQ ID NO: 19) |
| AIF1 | GGGGGCAAAATGGATAATTC | CTGTTTCTCTTCTGGGGACAG |
| | (SEQ ID NO: 20) | (SEQ ID NO: 21) |

The equivalent of 10 ng and 2 ng of cDNAs were used per gene as template for qPCR reactions with Power Sybr® green PCR Master Mix (Applied Biosystems). Each biological sample was subjected to the assay in triplicates per gene; Ct values were obtained with the ABI 7500 software (v.2.0.6). The comparative ΔΔCt method and the mitochondrial ATP6 gene have been used to determine the relative mRNA amount of each studied gene. The mitochondrial ATP6 gene has been used as normalizing gene since its mRNA steady-state levels remained almost unchanged in all the samples evaluated.

### 2.1.8 Mitochondria Extraction and Western Blotting Analysis

Thirty six retinas were isolated from 12 week-old mice (B6CBACa strain) and washed in PBS at 4° C.; they were then homogenized in extraction buffer (0.32 M sucrose, 30 mM Tris-HCl; pH 7.6, 5 mM MgAc, 100 mM KCl, 0.1% fatty acid-free BSA, 5 mM β-mercaptoethanol, and 1 mM PMFS) and mitochondria were purified as previously described for rat retinas (cf. FIG. 1 and example 1). 30 μg of mitochondrial proteins were treated with 150 μg/mL of Proteinase K (PK) in the presence or absence of 1% Triton X-100 (v/v) at 4° C. for 30 minutes. The reaction was stopped by addition of 1 mM PMFS (Sigma-Aldrich). Samples were collected by centrifugation at 10,000 g for 15 minutes at 4° C. Three independent mitochondria purifications were subjected to the analyses.

For whole proteins extracts, single retinas were homogenized in 50 μL of 20 mM HEPES and 60 mM mannitol (pH 7.2) using a 200 μL micro-hand-driven glass-glass potter at 4° C. Large cellular debris was spun down by a low speed centrifugation (1000 g for 5 minutes at 4° C.) and supernatants were used immediately for Western Blotting. Protein quantification was performed using the Bradford method (Bradford reagent from Sigma-Aldrich). After denaturation at 94° C. for 15 minutes, samples were resolved in 12% or 15% SDSPAGE and next transferred to a PVDF membrane. Membranes were probed with antibodies against NGB, AIF and ATP synthase subunit B (cf. Table 6). Immunoreactive bands were visualized with anti-mouse or anti-chicken coupled to horseradish peroxidase (0.1 mg/mL) followed by detection with Pierce® ECL Plus Western Blotting Substrate (Pierce, Thermo Scientific).

### 2.1.9 Tissue Homogenate Preparation and Respiratory Chain Enzyme Assays

Optic nerves were rapidly collected and kept frozen (−80° C.). Respiratory chain complex I and V enzymatic activities were measured using a Cary®50 UV-Vis spectrophotometer (Agilent technologies), as described for optic nerves from mice (Bouaita A, et al. (2012) Brain 135: 35-52) and each assay was made in triplicate. Complex I (CI) and Complex V (CV) values were converted to specific activities expressed as nanomoles of oxidized NADH/min/mg protein after protein quantification by the Bradford method. All chemicals were of the highest grade from Sigma-Aldrich.

### 2.1.10 Statistical Analyses

Values are expressed as means±SEM (Standard Error of the Mean). Statistical analyses were performed with the GraphPad Prism 6.0 software assuming a confidence interval of 95%. Generally, the observations within each group do not fit in a normal distribution, thus nonparametric methods have been applied for evaluating the significance. Data collected from control and Harlequin were compared using the unpaired non parametric significance test of Mann-Whitney (*≦0.05, **≦0.01 and ***≦0.005). Data collected from Harlequin treated eyes and untreated controlateral eyes were compared using the paired non parametric significance test of Wilcoxon (*≦0.05, **≦0.01 and ***≦0.005).

### 2.2 Results

### 2.2.1 Neuroglobin Expression in Control and Harlequin Mouse Retinas

It has been previously shown that NGB localizes to the mitochondria in rat retinas (cf. example 1). To examine the subcellular distribution of the NGB in adult mouse retinas, mitochondrial enriched fractions have been prepared by differential centrifugation and performed Western blot analysis (data not shown). Antibodies against NGB recognized three proteins with apparent molecular masses of about 17, 19 and 21 kDa in homogenates and mitochondria; in this latter the abundance is very high as observed for ATP synthase-β (a subunit of respiratory chain complex V). Interestingly, very discrete NGB signals were evidenced in: (i) the pellet (encompassing nuclei and unbroken cells); (ii) the high speed supernatant (obtained after the 10,000 g centrifugation which spun down mitochondria) indicating that NGB is enriched in the mitochondrial compartment. To further evaluate the amount of NGB which is translocated within the organelle, mild proteolysis with Proteinase K (PK) was performed.

The three forms of NGB and the ATP synthase-β gave strong signals indicating that these proteins were integrated into the mitochondria. Next, mitochondrial fractions were treated with PK in the presence of Triton X-100; the detergent disrupts mitochondrial membranes and leads to the entire proteolysis of mitochondrial proteins. ATP synthase-β and NGB protein signals were considerably diminished, confirming their localization inside the organelle in a protease-sensitive form (data not shown).

Therefore, NGB localizes to the mitochondria of mouse retinas as the inventors have previously shown in rat retinas.

To investigate, the abundance of NGB in RGCs, flat mounted retinas from 6 month-old mice were immunostained for NGB, BRN3A and the mitochondrial NDUFB6 protein, a complex I subunit (data not shown). BRN3A is a nuclear factor exclusively expressed by most of the RGCs in rodent retinas (Nadal-Nicolas et al., PLoS One. 2012; 7(11)); fluorescence microscopy of flat mounted retinas immunodetected for BRN3A showed many stained nuclei distributed throughout the retina. All the BRN3A-positive cells showed an intense NGB labeling as punctuate dots in the cytoplasm. In addition, some NGB positive-cells were not immunostained for BRN3A; they may correspond to displaced amacrin cells or astrocytes (data not shown). When antibodies against NGB and NDUFB6 were combined the majority of cells exhibited similar labeling patterns indicating some extent of colocalization between the two proteins (data not shown). Overall, signals appeared as strong punctuate fluorescent dots in the cytoplasm and apposed to the nuclei; thus distribution of both proteins in RGC bodies is comparable to the one described for mitochondria in mouse cells residing in the outer nuclear layer (ONL) (Johnson J E, et al. (2007) Mol Vis 13: 887-919).

NGB distribution in other cell populations was analyzed in radial cryosections of retinas immunostained for NGB in control and Hq mice aged 6 months (data not shown). In normal mice, it appears that the protein was particularly abundant in the ganglion cell layer (GCL) (data not shown) and the inner segments (IS) of photoreceptors (PRs), while very weak labeling with the NGB antibody was observed in PR outer segments (OS). The inner and outer plexiform layers (IPL and OPL) showed strong to moderate punctuate labeling; at the junction between the ONL and the OPL, the staining could represent the mitochondria in the synaptic terminals of PRs and in horizontal cells. The inner nuclear layer (INL) is usually divided in three regions; distal, middle and proximal, NGB immunostaining is strong in the three levels, especially in the distal region, some of the fluorescent cells were located at the very inner margin of the INL, they could be amacrine cells as previously described. Overall, NGB labeling in retinal neurons were consistent with the abundance of mitochondria in the different retinal compartments.

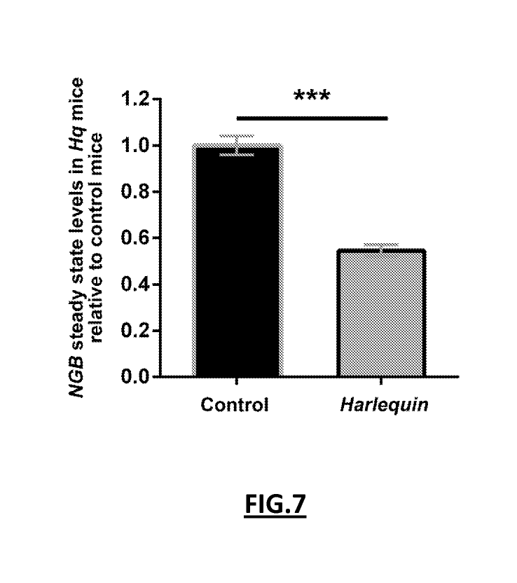

On the other hand, retinal sections from Hq mice showed a considerable reduction in the thickness of all the neuron layers with a significant diminution of NGB immunostaining (data not shown). In the GCL, the overall cell number is strongly reduced in Hq retina; as a consequence the number of NGB-positive cells was lower than in control retinas and the pattern of staining within one cell is less strong relative to the NGB-positive cells in control retinas (data not shown). To further investigate the reduction of NGB amount in Hq retinas, NGB mRNA steady-state levels in retinas from Hq mice was determined by RT-qPCR. Total RNA preparations from 37 Hq and 31 control retinas from 6 month-old mice were examined. The relative amount of NGB mRNA was 45% less abundant in Hq retina than in controls; the difference was significant (P<0.0001) (FIG. 7).

Whole protein extracts from Hq and control retinas were subjected to Western blotting analysis to corroborate this data. In whole extracts from control retinas the 17 kDa form gave the more intense signal; very weak signals from the 19 and 21 kDa was detected; indeed these NGB forms are enriched in mitochondrial fractions from both rat and mouse retinas (data not shown). Moreover, in the inventors' experience; the abundance of the three NGB forms varies in independent mitochondrial extractions (cf. example 1). This result can be explained by the highly flexible structure of the NGB which leads to great variations in protein conformation depending on exogenous ligand availability, pH changes or rupture/formation of the intra molecular disulfide bond.

In Hq retinas, NGB signals were strongly reduced compared to control retinas; as expected AIF was almost undetectable while ATP synthase-β amounts remained unchanged (data not shown). These results confirm the overall reduction of NBG in Hq retinas relative to age-matched controls, both at the level of mRNA and protein, thus the inventors has endeavor to re-establish NGB mRNA levels in Hq retinas to determine the impact on RGC integrity.

### 2.2.2 Design of a Gene Therapy Using Neuroglobin for Preventing Retinal Damage in Harlequin Mice

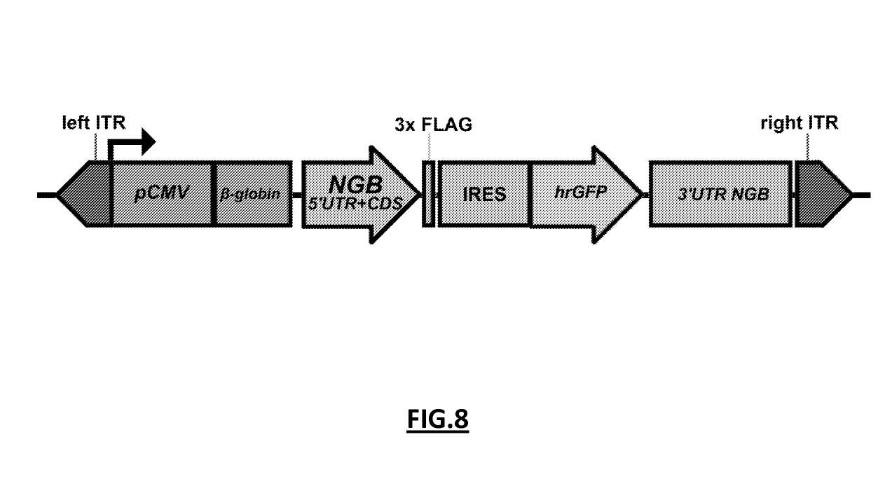

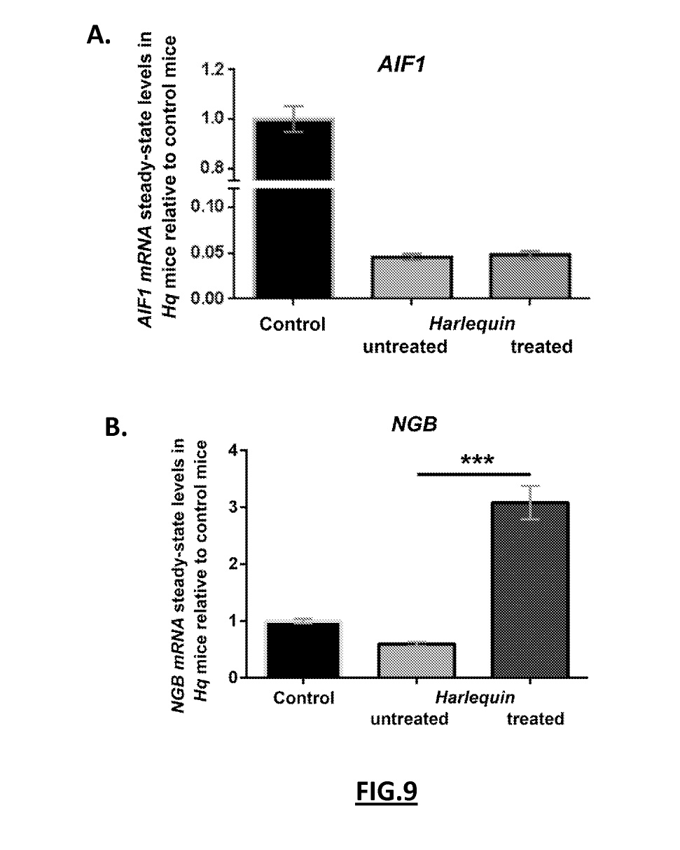

Ocular administration of recombinant AAV2 vectors did not produce adverse effects in mammals; in addition serotype 2 transduce highly efficiently inner retina, principally RGCs. Thus, a recombinant AAV2/2 possessing the mouse NGB ORF associated with the full-length 5′ and 3′ UTRs of the gene was constructed to ensure mRNA stability and translation capacity (FIG. 8). In an attempt to prevent RGC and optic nerve degeneration, a single intravitreal injection with the AAV2/2-NGB vector (2×10⁹ VG per eye) was performed in Hq mice 4-6 week-old; at this age the extent of RGC injury is minimal. Forty mice were evaluated within the course of this study; animals were euthanized between 5-6 months after vector administration. Since NGB sequences were inserted into the pAAV-hrGFP vector the GFP was used to qualitatively assess transduction efficiency (data not shown). The reconstruction of whole retinal sections from injected eye clearly shows a homogenous and intense GFP immunoreactivity mainly restricted to the GCL; On the contrary, the untreated eye displayed very little GFP labelling (data not shown). This data was strengthened when the NGB protein synthesized from the recombinant AAV2/2 vector was seeked in retinal sections using a specific antibody against the Flag epitope. Indeed, as shown in FIG. 8 the NGB ORF is in frame with the 3× FLAG® sequence at the C-terminus. In retinal sections from the AAV2/2-NGB treated eyes, many strong immunoreactive signals were only observed in the GCL while endogenous NGB staining in the other retinal layers was similar in Hq-treated and untreated eyes (data not shown). Hence, intravitreal administration of AAV2/2-NGB results in a highly efficient gene delivery to cells within the GCL, as previously described (Hellstrom M, et al. (2009) Gene Ther 16: 521-532). Next, the relative amount of the transduced NGB mRNA has been evaluated by real-time quantitative PCR in retinas from 14 Hq mice subjected to vector administration about 22 weeks before euthanasia (FIG. 9B). NGB mRNA transcribed from the recombinant AAV2/2 has been evidenced in total RNA preparations from retinas of injected eyes. The steady-state levels of the transduced NGB mRNA contributed to an approximate 3-fold increase of the overall amount of NGB mRNA relative to the one measured in control mouse retinas while no change was evidenced in the relative steady-state levels of AIF1 mRNA between retinas from treated and untreated eyes P=0.3 (FIG. 9A). Thus, AAV2/2-NGB administration to Hq eyes led to NGB overexpression essentially in resident GCL cells without adverse noticeable effects on mouse eyes up to 6 months.

2.2.3 Neuroglobin Overexpression Limits Gliosis Reaction in Retinas from Harlequin Mice

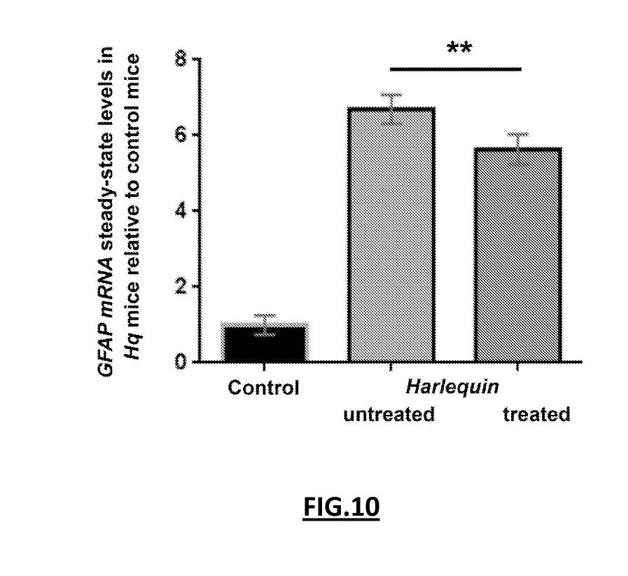

One prominent feature of the progressive retinal degeneration in Hq mice is glial cell activation, hence glial fibrillary acidic protein (GFAP) showed a significant increase, which begins in mice aged about 4 months. Mice older than 7 months exhibited a widespread GFAP immunoreactivity across the entire retinal thickness. To determine whether NGB overexpression could prevent the active growth of Müller cell processes, retinal sections from treated and untreated eyes were subjected to immunochemistry for GFAP and NGB. In control retinas GFAP immunofluorescence was confined exclusively to the GCL, corresponding to the end-feet of Müller cells and astrocytes resident in this cellular layer (data not shown). In Hq mice 6-7 month-old, GFAP immunoreactivity was markedly increased and was not just restricted to the GCL but also found in the Muller cell processes; intense GFAP-stained cell processes extended across the entire thickness of the retina from the untreated eye (data not shown). The steady-state levels of the GFAP mRNA also increased 6.7-fold relative to age-matched controls, confirming that an extensive glial response occurred in Hq retinas concomitantly with RGC degeneration (FIG. 10). In the retinal section from the AAV2/2-NGB treated eye, NGB labeling was noticeably enhanced in the GCL, substantiating the high transduction efficiency (data not shown). In opposition, GFAP immunoreactivity was distinctly less increased relative to controlateral untreated eyes, indeed the end-feet and processes of Müller cells were moderately labeled (data not shown). Moreover, the relative GFAP mRNA abundance between treated and untreated retinas diminished of 16% (FIG. 10), the difference was statistically significant (n=9, P=0.0039). Therefore, NGB overexpression hinders GFAP upregulation in Müller glia; this subtle change may be beneficial for RGC survival in AAV2/2-NGB treated eyes.

### 2.2.4 Retinal Ganglion Cell Loss in Harlequin Mice is Prevented by the Intravitreal Administration of AAV2/2-NGB

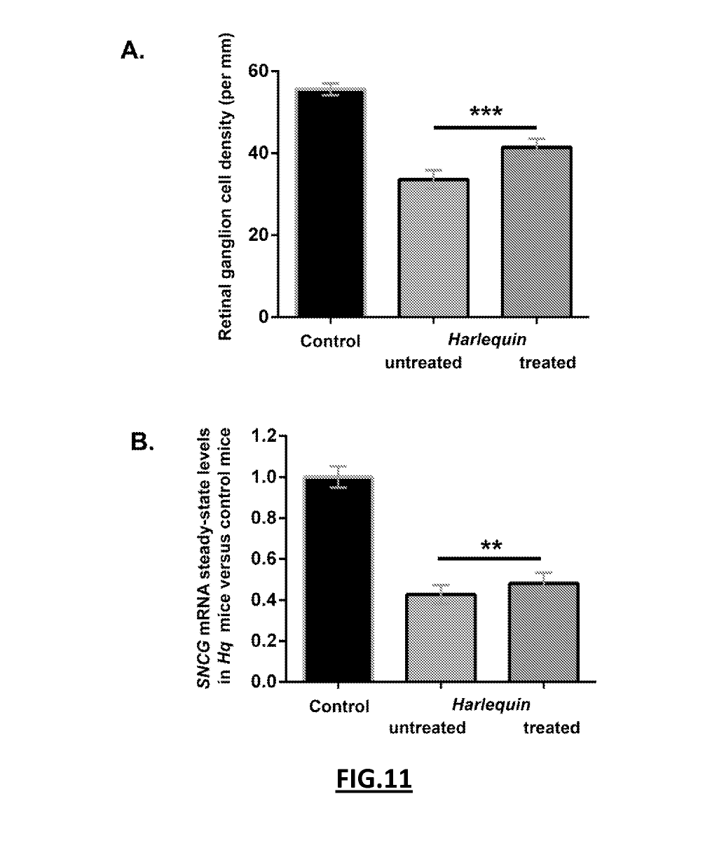

Retinal sections from Hq has been examined by immunochemistry using antibodies against NGB and BRN3A. Retinal sections of treated eye showed a noticeably increase of NGB immunostaining specifically in the GCL relative to the signals observed in the contralateral untreated eyes, many cells were BRN3A-positive (data not shown). More than 95% of the BRN3A-positive cells displayed a strong staining for NGB, hence, confirming the efficiency of AAV2/2-NGB on transducing RGCs. Retinal sections from the same animals were subjected to immunochemistry using antibodies against GFP and BRN3A (data not shown); the labeling obtained confirmed the high efficiency of vector transduction. Remarkably, the higher number of BRN3A-positive cells in the treated eye relative to the untreated one was noticeable as was that many of these cells were intensely GFP-labeled in their somas and processes. Besides, it was also visible GFP staining in the NFL and the INL; this latter can correspond to transduced Müller glial cells (data not shown). Hence, to corroborate the beneficial effect of NGB overexpression on RGC integrity, the quantification of RGC somas in retinal sections from Hq treated and untreated eyes has been proceeded subjected to immunostaining for BRN3A. The inventors have previously shown that by the age of 7-8 months Hq mice loss up to 36% of their overall RGC population (Bouaita A, et al. (2012) Brain 135: 35-52). Here the RGC density has been estimated in whole retinal sections for 24 control mice aged ˜7 months and compared to 13 Hq mice in which one eye was subjected to AAV2/2-NGB administration. The inventors confirmed that RGC somas were significantly reduced in retinas from Hq untreated eyes: 35±3/mm in Hq retinas relative to 58±2/mm in control retinas; a 40% diminution of the total amount of BRN3A-positive cells in the GCL has been observed (FIG. 11A). Uttermost, the number of RGCs in treated eyes per mm was 43±2; 24% higher than in contralateral untreated eyes from the same animals; these difference was statistically significant, P=0.0002 (FIG. 11A) and attained 75% of the number measured in control retinas.

Next, the relative amount of γ-synuclein (SNCG) mRNA has been determined by subjecting RNA preparations from 14 couples of Hq mouse retinas and 18 age-matched control retinas to RT-qPCR analysis. SNCG mRNA is considered as a very abundant molecule in adult mouse RGCs and it has been used as a marker of injured RGCs (Bouaita A, et al. (2012) Brain 135: 35-52). As previously shown, the steady-state level of SNCG mRNA was significantly diminished up to 43% of control value in Hq retinas from untreated eyes, P=0.0001 (FIG. 11B). In contrast, a consistent and statistically significant increase of 12.6% in its relative abundance was measured in treated eyes relative to the one measured in their contralateral untreated eyes (P=0.0004); although SNCG mRNA steady-state levels remained statistically different between treated eyes and controls, P=0.0002 (FIG. 11B).

Thus, intravitreal administration of AAV2/2-NGB prevented RGC loss and hindered SNCG downregulated expression in Hq retinas.

### 2.2.5 Neuroglobine Gene Overexpression Protected Nerve Fiber Integrity in Harlequin Mice

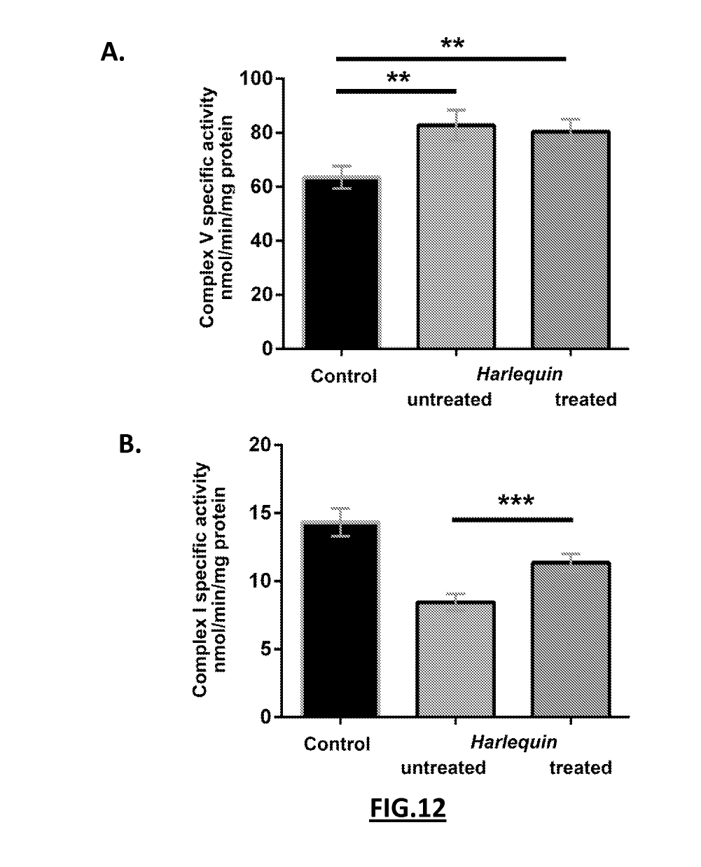

To substantiate that NGB overexpression in RGCs from Hq mice impeded, at a non negligible extent, their degeneration, both the amount of their axons as well as RCCI activity in optic nerves (ONs) have been evaluated. First, ON cross-sections were subjected to immunohistochemistry for the heavy chain (200 kDa) subunit of neurofilaments (NF200) to detect RGC axons. An obvious reduction of immunopositive dots in ONs from untreated Hq eyes relative to ONs isolated from age-matched control mice has been observed (data not shown). The axonal profiles detected in ON cross-sections from treated Hq eyes confirmed that they displayed a noticeably increase in NF200-immunopositive signals relative to untreated eyes (data not shown). This indicates that AAV2/2-NGB administration to Hq mouse eyes attenuates RGC axonal damage and corroborates data on their overall number preservation (FIG. 11A). In an attempt to establish a functional link between RGC number preservation and respiratory chain integrity, a spectrophotometric method for assessing respiratory chain complex activities has been utilized. This method has been successfully applied to accurately study respiratory chain in small amounts of tissues from Hq mice (Bouaita A, et al. (2012) Brain 135: 35-52); FIGS. 12A and 12B illustrate CV and CI activity measurements for 3 mouse groups: (1) 36 ONs from control mice aged 6-7 months; (2) 24 ONs from Hq eyes subjected to AAV2/2-NGB intravitreal injection and euthanized between 5 to 6 months after vector administration; (3) 24 ONs from the Hq contralateral untreated eyes. In a previous study on Hq mice, the inventors assessed respiratory chain function in ONs and they demonstrated that AIF depletion leads to a severe CI defect without affecting CV activity (Bouaita A, et al. (2012) Brain 135: 35-52). Here, the inventors show that CV activity was increased of about 22% in ONs from Hq relative to controls independently of AAV2/2-NGB treatment (FIG. 12A). Indeed, the difference between Hq values and controls was statistically significant (P=0.0006 and 0.0002 for untreated and treated eyes respectively) while the difference of CV activities in ONs from untreated and treated eyes was not significant (P=0.9).

Nonetheless, it was obvious from the current assessments that ONs from Hq untreated mice ˜7 month-old manifested a diminution in CI activity (expressed as nanomoles of oxidized NADH/min/mg protein) when compared to isogenic age-matched controls (FIG. 12B). The reduction was significant: 8.4±0.6 versus 14.8±1; 43% of the control value (P<0.0001). Noticeably, the ocular administration of AAV2/2-NGB protected efficiently CI function; indeed its enzymatic activity was statistically different from values obtained in ONs from untreated eyes (P<0.0001).

The specific activity of CI (11.6±0.6) attained in ONs from treated eyes 78% of the value measured in control mice; further, ONs from NGB-treated eyes exhibited 38% higher CI activity than ONs from their contralateral untreated eyes. Thus, high levels of NGB in RGCs from Hq mice rescued compromised CI activity; the inventors predict that this improvement was involved in the RGC robustness evidenced (FIG. 11).

### 2.2.6 Sustained Preservation of Nerve Fibers, Due to Neuroglobin Gene Overexpression, Confers Improvements in Harlequin Mouse Vision

To make the most trustworthy proof-of-concept on the protective effect of NGB against RGC degeneration besides indications gathered on cell loss prevention and CI activity protection it is also required to confirm the presence of fibers bundles in eyes fundus and their ability to transfer visual inputs to the visual cortex. Hence, a thorough evaluation of eye fundus has been first performed using Confocal Scanning Laser Ophthalmoscopy (cSLO), a reliable method for in vivo cellular imaging in the retina (Paques M, et al. (2006) Vision Res 46: 1336-1345). Imaging of the nerve fiber layer (NFL) was facilitated by the use of pigmented mice and the high contrast between the fiber bundles (RGC axon packages) and the dark background. Striations of NFL radiating from the optic disc were clearly visible in each eye from mouse before AAV2/2-NGB administration for all Hq mice 6 week-old (data not shown). Subsequently, each area of the eye fundus (nasal, temporal, inferior and superior) was visualized monthly until euthanasia to follow over time nerve fiber disappearance and seek for any change related to NGB overexpression.

Six months after vector administration, a substantial loss of nerve fiber bundles was evidenced in almost all the untreated eyes; in general we can observe that about half of the entire retinal surface gave the impression of being devoid of fibers that could be either in the temporal or nasal areas. In contrast, images collected for more than the half of eyes that received AAV2/2-NGB revealed significantly well preserved axon tracks in all the areas visualized. Up to date 40 mice have been extensively evaluated using cSLO; more than 80% of the untreated eyes did reveal RGC axon degeneration in mice from the age of 4 months; while 22 eyes out of the 40 subjected to AAV2/2-NGB treatment exhibited high densities of fiber bundles in all the areas examined up to 6 months after vector administration. Thus; NGB overexpression efficiently protected Hq mice against optic nerve degeneration.

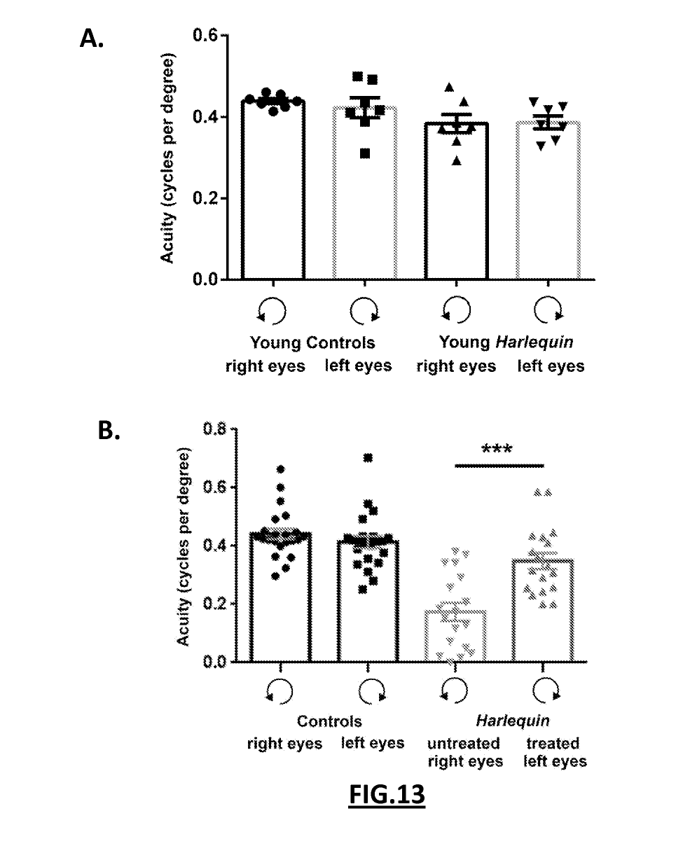

Ultimately, to address the overriding question of whether AAV2/2-NGB administration confers improvements in vision to Hq mice, visual function of young animals has been assessed at different times after gene therapy by studying their optomotor responses to rotating sinusoidal gratings (OptoMotry™). Visual acuities were meticulously measured in isogenic age-matched controls and Hq mice to gather precise thresholds (highest spatial frequency each mouse could track) under our experimental conditions. Tracking capability was examined in both clockwise and counter clockwise drum rotations at different frequencies because only temporal-to-nasal motion is effective through each eye, clockwise movement will drive tracking through the left eye, whereas counterclockwise motion will activate the right eye. Optomotor responses recorded from Hq mice aged 4-8 weeks (before any ocular intervention) and wild-type mice of the same age were similar: for the clockwise (left eye sensitivity) and the counterclockwise (right eye sensitivity) pattern of rotations; indeed values indicated no significant difference: P=0.06 and 0.44 respectively (FIG. 13A). This data suggests that visual function of Hq mice was not compromised during the first weeks of life and is in agreement with cSLO examinations in which fiber disappearance was noticeable in Hq mice aged 4 months or older (data not shown). Next the evaluation of Hq mice which received in their left eyes AAV2/2-NGB has been proceeded; they were subjected to the test twice, at about 3 months post-injection and just before euthanasia (up to 6 months post-injection). FIG. 13B illustrates clockwise and couterclockwise visual acuities from Hq mice and age-matched control mice. Very reduced head-tracking behavior (counterclockwise responses) was observed in the untreated eyes of Hq mice aged of ˜7 months; indeed Hq mice visual acuity from the left eyes (n=18) was significantly poorer than control mice (n=22): 0.175±0.03 versus 0.44±0.018 cycles per degree (P<0.0001). Impressively, a visual-acuity threshold (clockwise response) of 0.35±0.03 cycles per degree was recorded in Hq treated eyes; the decrease in visual performance in the untreated eyes (counterclockwise responses) was evidenced at the age of 4-5 months; though the responses recorded from the treated-eyes remained stable until euthanasia (FIG. 13B). Visual acuity in Hq treated eyes was significant different from the one measured in untreated eyes (P<0.0001); and represents a value 99% superior to the one from untreated eyes. Thus, this data confirms that NGB overexpression benefit encompassed the long-lasting protection of RGCs and their axons along with the maintenance of their functional integrity.

1. A neuroglobin agonist for use in the treatment or prevention of a mitochondrial disease associated with respiratory chain complex I (RCCI) deficiency and/or respiratory chain complex III (RCCIII) deficiency.

2. The neuroglobin agonist for the use of claim 1, wherein said mitochondrial disease is associated with Apoptosis Inducing Factor (AIF) deficiency.

3. The neuroglobin agonist for the use of claim 1 or 2, wherein said mitochondrial disease is not associated with a mutation or deletion of neuroglobin gene or is not induced by neuroglobin deficiency.

4. The neuroglobin agonist for the use according to any one of claims 1 to 3, for restoring or improving RCCI and/or RCCIII function.

5. The neuroglobin agonist for the use according to any one of claims 1 to 4, wherein said mitochondrial disease associated with RCCI deficiency and/or RCCIII deficiency is a neurodegenerative disease or an ocular disease.

6. The neuroglobin agonist for the use of claim 5, wherein said mitochondrial disease associated with RCCI deficiency and/or RCCIII deficiency is a retinal disease.

7. The neuroglobin agonist for the use according to any one of claims 1 to 6, wherein said neuroglobin agonist is a polypeptide selected from the group consisting of a dominant activated mutant of neuroglobin, a wild-type neuroglobin protein, a fragment and a peptidomimetic thereof.

8. The neuroglobin agonist for the use of claim according to any one of claims 1 to 6, wherein said neuroglobin agonist is a nucleic acid which comprises an expression cassette comprising a polynucleotide encoding neuroglobin protein, said polynucleotide being operatively linked to at least one transcriptional regulatory sequence.

9. The neuroglobin agonist for the use of claim 8, wherein said at least one transcriptional regulatory sequence comprises 3′UTR and/or 5′UTR neuroglobin sequence.

10. The neuroglobin agonist for the use of claim 8 or 9, wherein said at least one transcriptional regulatory sequence is one or more of a neuroglobin promoter, a retina specific promoter or a central nervous system specific promoter.

11. The neuroglobin agonist according to any one of claims 8 to 10, wherein said expression cassette is inserted into an expression vector.

12. The neuroglobin agonist for the use of claim 11, wherein said expression vector is a viral vector.

13. The neuroglobin agonist for the use of claim 12, wherein said expression vector is an adeno-associated virus vector.

14. A method for preventing or treating a mitochondrial disease associated with respiratory chain complex I (RCCI) deficiency and/or respiratory chain complex III (RCCIII) deficiency in a subject having or at risk of having such a disorder, comprising administration of a therapeutically effective amount of a neuroglobin agonist to the subject.

15. The method of claim 14, wherein said mitochondrial disease is associated with Apoptosis Inducing Factor (AIF) deficiency.

16. The method of claim 14 or 15, wherein said mitochondrial disease is not associated with a mutation or deletion of neuroglobin gene or is not induced by neuroglobin deficiency.

17. The method according to any one of claims 14 to 16, wherein said neuroglobin agonist is a nucleic acid which comprises an expression cassette comprising a polynucleotide encoding neuroglobin protein, said polynucleotide being operatively linked to at least one transcriptional regulatory sequence and wherein said method further comprises the step of expressing said polynucleotide in said subject.

18. The method according to any one of claims 14 to 16, wherein said neuroglobin agonist is a polypeptide selected from the group consisting of a dominant activated mutant of neuroglobin, a wild-type neuroglobin protein, a fragment and a peptidomimetic thereof.


SEQUENCE LISTING (33 sequences)

SEQ ID NO: 1 - protein, 151 aa, Homo sapiens
MERPEPELIRQSWRAVSRSPLEHGTVLFARLFALEPDLLPLFQYNCRQFSSPEDCLSSPEFLDHIRKVMLVIDAAVTNVEDLSSLEEYLASLGRKHRAVGVKLSSFSTVGESLLYMLEKCLGPAFTPATRAAWSQLYGAVVQAMSRGWDGE

SEQ ID NO: 2 - DNA, 1885 bp, Homo sapiens
ttcccaggccaccatagcggctggcggagggagcgcgcgccttgctggcctggagggggcgggggccgtggcggctttaaagcgcccagcccaggcgtcgcggggtggggcggctctggcggctgcggggcgcagggcgcagcggccaagcggggtccccggaagcacagctggggtgtctccacctacgactggccgcgcgccttttctctcccgcgccagggaaggagcggctgcggcccccgccgggcggaggcacggggggcgtacgaggggcggaggggaccgcgtcgcggaggagatggcgcggcacgtgcggtgacggcacccgagccctgagggtcccagccccgcgctccgcgtccccgggacagcatggagcgcccggagcccgagctgatccggcagagctggcgggcagtgagccgcagcccgctggagcacggcaccgtcctgtttgccaggctgtttgccctggagcctgacctgctgcccctcttccagtacaactgccgccagttctccagcccagaggactgtctctcctcgcctgagttcctggaccacatcaggaaggtgatgctcgtgattgatgctgcagtgaccaatgtggaagacctgtcctcactggaggagtaccttgccagcctgggcaggaagcaccgggcagtgggtgtgaagctcagctccttctcgacagtgggtgagtctctgctctacatgctggagaagtgtctgggccctgccttcacaccagccacacgggctgcctggagccaactctacggggccgtagtgcaggccatgagtcgaggctgggatggcgagtaagaggcgaccccgcccggcagcccccatccatctgtgtctgtctgttggcctgtatctgttgtagcccaggctccccaagcttccctgcatcttggtccttgtccccttggccacactggagaggtgatggggcagggctgggtctcagtatcctagagtccagctgcagaaggagtggcttttcctccaggaaggggcttctgggtgtcccctcatccccagtagcctctttcttgcgtttctttttaccttttttggcactccctctgaccccgcgatgagtgttttggtggcagaggtgggatgagctggaaaggtatggaggtgggagaggatggggctcttctgtctgtcctgcttcttcaggtgagtgcaggccaaggcgggggtgagatggctgagcttccagcgccttctgtcctgcctgcccagtcccttcactgctttcctgccccaagatggcttgcttttcacaaataaagagaaagagcagctttagccttcttggtggaatcccaggcagtgggagcagaatcagaactgccagggaagggaagggggacctgggtctcaatgggtctcatttgagtctcgcgggctgtgcagatgccctgacagagtcggtttcctttggcggcattccctttccctcattcagcacttctgctgggaactccctgactattccgctgctgcaggaacccagctagctggccaggtggggaggggctggggaccggccaggaaggaggggtgacttcatcccagagagacccgagttcccccagcccttcatcaccaacccgctcctgcaggagtgagtcttacctcccctggccctcctttctggctcagcctgcagcgactgtgaggccacagctcctcagattcactgcccgctgtgtgccagtactcaggcagctggagagaagagaaggcagcagcagaggcccccgccctcaccccagccatctgcacttgtaccatttgctctgtgctgactgtggtcctataaattcatgagaaataaactggttctgtgtgcaaaaaaaaa

SEQ ID NO: 3 - DNA, 1054 bp, Homo sapiens
gaggcgaccccgcccggcagcccccatccatctgtgtctgtctgttggcctgtatctgttgtagcccaggctccccaagcttccctgcatcttggtccttgtccccttggccacactggagaggtgatggggcagggctgggtctcagtatcctagagtccagctgcagaaggagtggcttttcctccaggaaggggcttctgggtgtcccctcatccccagtagcctctttcttgcgtttctttttaccttttttggcactccctctgaccccgcgatgagtgttttggtggcagaggtgggatgagctggaaaggtatggaggtgggagaggatggggctcttctgtctgtcctgcttcttcaggtgagtgcaggccaaggcgggggtgagatggctgagcttccagcgccttctgtcctgcctgcccagtcccttcactgctttcctgccccaagatggcttgcttttcacaaataaagagaaagagcagctttagccttcttggtggaatcccaggcagtgggagcagaatcagaactgccagggaagggaagggggacctgggtctcaatgggtctcatttgagtctcgcgggctgtgcagatgccctgacagagtcggtttcctttggcggcattccctttccctcattcagcacttctgctgggaactccctgactattccgctgctgcaggaacccagctagctggccaggtggggaggggctggggaccggccaggaaggaggggtgacttcatcccagagagacccgagttcccccagcccttcatcaccaacccgctcctgcaggagtgagtcttacctcccctggccctcctttctggctcagcctgcagcgactgtgaggccacagctcctcagattcactgcccgctgtgtgccagtactcaggcagctggagagaagagaaggcagcagcagaggcccccgccctcaccccagccatctgcacttgtaccatttgctctgtgctgactgtggtcctataaattcatgagaaataaactggttctgtgtgcaaaaaaaaa

SEQ ID NO: 4 - DNA, 375 bp, Homo sapiens
ttcccaggccaccatagcggctggcggagggagcgcgcgccttgctggcctggagggggcgggggccgtggcggctttaaagcgcccagcccaggcgtcgcggggtggggcggctctggcggctgcggggcgcagggcgcagcggccaagcggggtccccggaagcacagctggggtgtctccacctacgactggccgcgcgccttttctctcccgcgccagggaaggagcggctgcggcccccgccgggcggaggcacggggggcgtacgaggggcggaggggaccgcgtcgcggaggagatggcgcggcacgtgcggtgacggcacccgagccctgagggtcccagccccgcgctccgcgtccccgggacagc

SEQ ID NO: 5 - DNA, 1630 bp, Mus musculus
gccaccgtagctttaatgggcggttctctgggagcttcggggtgtagggcgcagctgcccaagcggggtccccggaagcgcccgaggccaagctggccgtgcgcatcctctgggctggaccagtaaaggcgcagttgctgggccccacccaactaaggcactgggcctggaggggggctcccccgcgtcgccgaggagatggcgctgcatgtgcgttgactgcacccacgcctcgagggtcccatcactgcgtcccgcgagtctcctgggagagagagcatggagcgcccggagtcagagctgatccggcagagctggcgggtagtgagccgcagccctctggaacatggcactgtcctgttcgccaggctcttcgccctggaacccagcctgctgcctctcttccagtacaatggccgccagttctccagccctgaggactgtctctcctctccagaattcctggaccacattaggaaggtgatgctagtgattgatgctgcagtgaccaacgtggaggacctgtcttcattggaggagtacctgaccagcttgggcaggaagcatcgggcagtgggagtgaggctcagctccttctcgacagtaggcgagtccctgctctacatgctggagaagtgcctgggtcccgactttacaccagctacaaggaccgcctggagccgactctacggagctgtggtgcaagccatgagccgaggctgggatggggagtaagagacgagccagtgcccctatctatgtgtgtctgtctgttgatctgcctgttgtagtcttagcctctcccccagggtctctctataccttggtcctcatccccttggccatcctagagaggtgatgggagagggctctgtctcagcttctggtcagctgcagggaggtggatttccttccctcatccccatagatgtcctgctgcatgcctggttagctcgccccaggaggcagaacaatttgggtagtgaggtggcatgggggttcttctcagagaagctttctgccaaggtgagggaaaagtgactaggcttctagaattttccaccttgcctgcccagtctctctgatacttccctgccaagctgtttactttccaacaatgaagaggagagactttttagtgtttctcaggcaagggaagcatagaagatggcattcttcagtggcattgggcagtggctcagctgctcagacgccccatacaaaacctcttgtgctcttggttcagcatttctggtggaagcaccccggtattcttttgggggaaacctaactggccagccataatggagaggccagaagggcaaggaggaggagtgacctcatcccaaatggcatggaattctggggctcttcctgccacttaattgtagaagggctgaatcctgtctccccaccctcttctgctcacccaccgctctccagctgtgggcccctagctctatgtttaatgcttgaggtgtactcgtgcctgggctgatgagtgggagagaatagagtagggagggcccttgtacccatccccagccacccacactttcttgctctgtgctgtggctctataaactcacaagaaataaacatgttctgtgtgctgaaaaaaaaaaaaaaa

SEQ ID NO: 6 - DNA, 279 bp, Mus musculus
gccaccgtagctttaatgggcggttctctgggagcttcggggtgtagggcgcagctgcccaagcggggtccccggaagcgcccgaggccaagctggccgtgcgcatcctctgggctggaccagtaaaggcgcagttgctgggccccacccaactaaggcactgggcctggaggggggctcccccgcgtcgccgaggagatggcgctgcatgtgcgttgactgcacccacgcctcgagggtcccatcactgcgtcccgcgagtctcctgggagagagagc

SEQ ID NO: 7 - DNA, 895 bp, Mus musculus
gagacgagccagtgcccctatctatgtgtgtctgtctgttgatctgcctgttgtagtcttagcctctcccccagggtctctctataccttggtcctcatccccttggccatcctagagaggtgatgggagagggctctgtctcagcttctggtcagctgcagggaggtggatttccttccctcatccccatagatgtcctgctgcatgcctggttagctcgccccaggaggcagaacaatttgggtagtgaggtggcatgggggttcttctcagagaagctttctgccaaggtgagggaaaagtgactaggcttctagaattttccaccttgcctgcccagtctctctgatacttccctgccaagctgtttactttccaacaatgaagaggagagactttttagtgtttctcaggcaagggaagcatagaagatggcattcttcagtggcattgggcagtggctcagctgctcagacgccccatacaaaacctcttgtgctcttggttcagcatttctggtggaagcaccccggtattcttttgggggaaacctaactggccagccataatggagaggccagaagggcaaggaggaggagtgacctcatcccaaatggcatggaattctggggctcttcctgccacttaattgtagaagggctgaatcctgtctccccaccctcttctgctcacccaccgctctccagctgtgggcccctagctctatgtttaatgcttgaggtgtactcgtgcctgggctgatgagtgggagagaatagagtagggagggcccttgtacccatccccagccacccacactttcttgctctgtgctgtggctctataaactcacaagaaataaacatgttctgtgtgctgaaaaaaaaaaaaaaa

SEQ ID NO: 8 - DNA, 454 bp, Mus musculus
atggaaagacctgaaagtgaactcattagacagtcctggagagtcgtgtcaagatcacctctggagcatggaaccgtcctgtttgcccggctgttcgctctcgagcccagcctgctccctctgttccagtacaacggacgccagtttagctcccctgaggactgcctgtctagtccagaatttctggatcacatcagaaaagtgatgctggtcattgacgccgctgtgactaatgtcgaggacctgtcaagcctcgaggaatacctgacctctctcggcaggaagcatagagcagtgggagtccgactgtcctctttcagtacagtgggggagtccctgctctatatgctggaaaaatgtctcggtccagactttacccccgccaccagaacagcttggtcacggctgtatggggcagtggtccaggctatgtcacgaggttgggatggcgaac

SEQ ID NO: 9 - DNA, 7255 bp, Artificial sequence
  pAAV2-NGB vector full-length sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgataattaaccctcactaaagggaacaaaagctggagctccaccgcggtggcggccgctctagcccgggcggatccgccaccgtagctttaatgggcggttctctgggagcttcggggtgtagggcgcagctgcccaagcggggtccccggaagcgcccgaggccaagctggccgtgcgcatcctctgggctggaccagtaaaggcgcagttgctgggccccacccaactaaggcactgggcctggaggggggctcccccgcgtcgccgaggagatggcgctgcatgtgcgttgactgcacccacgcctcgagggtcccatcactgcgtcccgcgagtctcctgggagagagagcatggaaagacctgaaagtgaactcattagacagtcctggagagtcgtgtcaagatcacctctggagcatggaaccgtcctgtttgcccggctgttcgctctcgagcccagcctgctccctctgttccagtacaacggacgccagtttagctcccctgaggactgcctgtctagtccagaatttctggatcacatcagaaaagtgatgctggtcattgacgccgctgtgactaatgtcgaggacctgtcaagcctcgaggaatacctgacctctctcggcaggaagcatagagcagtgggagtccgactgtcctctttcagtacagtgggggagtccctgctctatatgctggaaaaatgtctcggtccagactttacccccgccaccagaacagcttggtcacggctgtatggggcagtggtccaggctatgtcacgaggttgggatggcgaacgtcgactcgaggactacaaggatgacgatgacaaggattacaaagacgacgatgataaggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccatggtgagcaagcagatcctgaagaacaccggcctgcaggagatcatgagcttcaaggtgaacctggagggcgtggtgaacaaccacgtgttcaccatggagggctgcggcaagggcaacatcctgttcggcaaccagctggtgcagatccgcgtgaccaagggcgcccccctgcccttcgccttcgacatcctgagccccgccttccagtacggcaaccgcaccttcaccaagtaccccgaggacatcagcgacttcttcatccagagcttccccgccggcttcgtgtacgagcgcaccctgcgctacgaggacggcggcctggtggagatccgcagcgacatcaacctgatcgaggagatgttcgtgtaccgcgtggagtacaagggccgcaacttccccaacgacggccccgtgatgaagaagaccatcaccggcctgcagcccagcttcgaggtggtgtacatgaacgacggcgtgctggtgggccaggtgatcctggtgtaccgcctgaacagcggcaagttctacagctgccacatgcgcaccctgatgaagagcaagggcgtggtgaaggacttccccgagtaccacttcatccagcaccgcctggagaagacctacgtggaggacggcggcttcgtggagcagcacgagaccgccatcgcccagctgaccagcctgggcaagcccctgggcagcctgcacgagtgggtgtaatagggtaccaggtaagtgtacccaattcgccctatagtgagtcgtattagatctgagacgagccagtgcccctatctatgtgtgtctgtctgttgatctgcctgttgtagtcttagcctctcccccagggtctctctataccttggtcctcatccccttggccatcctagagaggtgatgggagagggctctgtctcagcttctggtcagctgcagggaggtggatttccttccctcatccccatagatgtcctgctgcatgcctggttagctcgccccaggaggcagaacaatttgggtagtgaggtggcatgggggttcttctcagagaagctttctgccaaggtgagggaaaagtgactaggcttctagaattttccaccttgcctgcccagtctctctgatacttccctgccaagctgtttactttccaacaatgaagaggagagactttttagtgtttctcaggcaagggaagcatagaagatggcattcttcagtggcattgggcagtggctcagctgctcagacgccccatacaaaacctcttgtgctcttggttcagcatttctggtggaagcaccccggtattcttttgggggaaacctaactggccagccataatggagaggccagaagggcaaggaggaggagtgacctcatcccaaatggcatggaattctggggctcttcctgccacttaattgtagaagggctgaatcctgtctccccaccctcttctgctcacccaccgctctccagctgtgggcccctagctctatgtttaatgcttgaggtgtactcgtgcctgggctgatgagtgggagagaatagagtagggagggcccttgtacccatccccagccacccacactttcttgctctgtgctgtggctctataaactcacaagaaataaacatgttctgtgtgctgaaaaaaaaaaaaaaacggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 20 bp, Artificial sequence
  Primer, probe
ctcaggcaagggaagcatag

SEQ ID NO: 11 - DNA, 20 bp, Artificial sequence
  Primer, probe
cagttaggtttcccccaaaa

SEQ ID NO: 12 - DNA, 20 bp, Artificial sequence
  Primer
aggctatgtcacgaggttgg

SEQ ID NO: 13 - DNA, 20 bp, Artificial sequence
  Primer
gggtaaccctatgcagtcgt

SEQ ID NO: 14 - DNA, 21 bp, Artificial sequence
  Primer
cgtaattacaggcttccgaca

SEQ ID NO: 15 - DNA, 20 bp, Artificial sequence
  Primer
agctgtaagccggactgcta

SEQ ID NO: 16 - DNA, 19 bp, Artificial sequence
  Primer
ggaggcagctgagaagacc

SEQ ID NO: 17 - DNA, 20 bp, Artificial sequence
  Primer
actgtgttgacgctgctgac

SEQ ID NO: 18 - DNA, 20 bp, Artificial sequence
  Primer
cccgttctctggaagacact

SEQ ID NO: 19 - DNA, 20 bp, Artificial sequence
  Primer
cttcagggctgagagcagtc

SEQ ID NO: 20 - DNA, 20 bp, Artificial sequence
  Primer
gggggcaaaatggataattc

SEQ ID NO: 21 - DNA, 21 bp, Artificial sequence
  Primer
ctgtttctcttctggggacag

SEQ ID NO: 22 - RNA, 19 nt, Artificial Sequence
  siRNA
gugagucccugcucuacau

SEQ ID NO: 23 - RNA, 19 nt, Artificial sequence
  siRNA
gccacacgauugcugucuu

SEQ ID NO: 24 - DNA, 20 bp, Artificial sequence
  Primer
ccaacgatgaaggagagagg

SEQ ID NO: 25 - DNA, 20 bp, Artificial sequence
  Primer
cagaaatgccgaaccaagag

SEQ ID NO: 26 - DNA, 21 bp, Artificial sequence
  Primer
caaccaaccttctagggcttc

SEQ ID NO: 27 - DNA, 20 bp, Artificial sequence
  Primer
gcggtaagaagtgggctaaa

SEQ ID NO: 28 - DNA, 20 bp, Artificial sequence
  Primer
gtaacctcggtggctgagaa

SEQ ID NO: 29 - DNA, 20 bp, Artificial sequence
  Primer
ttccaagtcctccttgcgta

SEQ ID NO: 30 - DNA, 20 bp, Artificial sequence
  Primer
aggcctattttgccgtacaa

SEQ ID NO: 31 - DNA, 20 bp, Artificial sequence
  Primer
cgtctcacaccctcctcagt

SEQ ID NO: 32 - DNA, 20 bp, Artificial sequence
  Primer
gactgacttgctccggaaag

SEQ ID NO: 33 - DNA, 20 bp, Artificial sequence
  Primer
gtctgaagtgagcgggtgag